SEQUENCE LISTING (9 sequences)

SEQ ID NO: 1 - RNA, 40 nt, synthetic construct
  source 1..40: mol_type = other RNA
tctgcacgttcgaataagtctccggtgtttcgagaccctt

SEQ ID NO: 2 - RNA, 40 nt, synthetic construct
  source 1..40: mol_type = other RNA
caatgtttgactgtacatgcggaaatttggaccctcgaag

SEQ ID NO: 3 - RNA, 40 nt, synthetic construct
  source 1..40: mol_type = other RNA
ccctacggactggactagcacatgacagttagccattaag

SEQ ID NO: 4 - RNA, 40 nt, synthetic construct
  source 1..40: mol_type = other RNA
tcagttggcactatagccatacccttagaaatgcaacgtt

SEQ ID NO: 5 - RNA, 40 nt, synthetic construct
  source 1..40: mol_type = other RNA
ggtacccgttgacacattgtaatttccagagatttgacac

SEQ ID NO: 6 - RNA, 31 nt, synthetic construct
  source 1..31: mol_type = other RNA
tcgaataagtctccggtgtttcgagaccctt

SEQ ID NO: 7 - RNA, 26 nt, synthetic construct
  source 1..26: mol_type = other RNA
taagtctccggtgtttcgagaccctt

SEQ ID NO: 8 - RNA, 23 nt, synthetic construct
  source 1..23: mol_type = other RNA
tctgcacgttcgaataagccctt

SEQ ID NO: 9 - RNA, 34 nt, synthetic construct
  source 1..34: mol_type = other RNA
tctgcacgttcgaataagtcgccgcgagaccctt

(19) United States
(12) Patent Application Publication
(10) Pub. No.: US 2025/0297264 A1
(43) Pub. Date: Sep. 25, 2025

(54) RNA APTAMER TARGETING OF ADAM8 IN CANCER GROWTH AND METASTASIS

(71) Applicant: University of South Florida, Tampa, FL (US)

(72) Inventors: Paul C. Kuo, St. Petersburg, FL (US); Zhiyong Mi, Tampa, FL (US)

(73) Assignee: University of South Florida, Tampa, FL (US)

(21) Appl. No.: 19/230,982

(22) Filed: Jun. 6, 2025

Related U.S. Application Data
(63) Continuation of application No. PCT/US2023/082515, filed on Dec. 5, 2023.
(60) Provisional application No. 63/386,269, filed on Dec. 6, 2022.

(51) Int. Cl. C12N 15/115; A61P 35/00

(52) U.S. Cl. CPC C12N 15/115; A61P 35/00; C12N 2310/16; C12N 2310/531

(57) ABSTRACT

A novel composition and method of treating cancer is described herein. The novel composition is comprised of an RNA aptamer directed to binding Adam8 to decrease expression. The RNA aptamer may be Apt-1 or Apt-1-26nt. Administration of the RNA aptamer exhibited decreased cancer cell growth and metastasis in cancers associated with increased expression of Adam8.

### CROSS REFERENCE TO RELATED APPLICATIONS

This application is a continuation of and claims priority to International Application Serial No. PCT/US2023/082515, entitled “RNA Aptamer Targeting of ADAM8 in Cancer Growth and Metastasis”, filed Dec. 5, 2023, which claims priority to U.S. Provisional Patent Application Ser. No. 63/386,269, entitled “Aptamer Targeting A Disintegrin and Metalloproteinase Domain-Containing Protein 8”, filed Dec. 6, 2022, the contents of each of which are hereby incorporated by reference into this disclosure.

### SEQUENCE LISTING

The sequence listing entitled “RNA Aptamer Targeting of Adam8 in Cancer Growth and Metastasis” in XML format, created on Nov. 26, 2023, and being 9000 bytes in size, is hereby incorporated by reference into this disclosure.

### FIELD OF INVENTION

This invention relates to treatment and/or prevention of cancers. Specifically, the invention provides a novel RNA aptamer targeting Adam8 and associated method of use to treat cancers by blocking Adam8, inhibiting cancer cell growth and metastasis, and reversing cancer-derived osteopontin-induced myofibroblast cancer-associated fibroblast phenotype (myCAF).

### BACKGROUND OF THE INVENTION

The growth and metastasis of cancer cells are regulated by reciprocal cross talk between the tumor microenvironment (TME) and cancer stem cells. The TME consists of highly complex and dynamic molecules, blood vessels, and various other cell types, which surround the cancer cell. One key component of the TME is the cancer-associated fibroblast (CAF), which executes multiple functions in order to manipulate cancer development, including facilitating extracellular matrix remodeling, accelerating angiogenesis, promoting the epithelial-mesenchymal transition of cancer stem cells, increasing cancer cells invasion and metastasis, and inducing the ability of tumor cells to evade immunosurveillance and develop resistance to chemotherapies. Molecular drivers that originate from, and are involved in, the TME-cancer stem cell interaction network are ideal targets in either diagnostic or in therapeutic clinical practice.

Adam8 is a transmembrane glycoprotein that is selectively expressed and induced by a variety of inflammatory stimuli¹. It comprises 824 amino acids with a prototypical N-terminal prodomain; a metalloproteinase-, disintegrin- and cysteine-rich, epidermal-growth factor-like transmembrane domain; and a cytoplasmic tail². Upon autocatalytic prodomain removal, the 90 kDa active form of Adam8 is processed to release a 30 kDa soluble metalloproteinase domain resulting in a 60 kDa remnant on the cell surface³. While the expression of Adam8 under normal circumstances is minimal, Adam8 can be upregulated in a variety of pathologic conditions, including asthma, liver injury, and, most notably, cancer⁴. Increased expression of Adam8 has been correlated with enhanced tumor growth and metastasis in breast, pancreatic, liver, colon, and renal (kidney) cancers, among others. However, the mechanism by which Adam8 abets cancer potentiation is unknown.

RNA aptamers are small-structured single-stranded RNAs that are established alternatives to antibody-based therapy for the treatment of cancer⁵⁻⁸. RNA aptamers bind to their target proteins with high affinity, are quite stable, and lack immunogenicity. Aptamers are developed by means of an iterative selection method termed systematic evolution of ligands by exponential enrichment (SELEX). The shed 30 kDa soluble metalloproteinase domain of Adam8 represents an ideal target for RNA-aptamer-mediated inhibition⁹.

The inventors found that targeting Adam8 in the extracellular space using RNA aptamer technology can inhibit the growth and metastasis of cancer cells. MDA-MB-231 human breast cancer and HepG2 human liver cancer cell lines were used to characterize the pharmacokinetic and pharmacodynamic properties of an Adam8-directed RNA aptamer (Adam8-Apt-1-26nt) and demonstrate its effect on in vitro and in vivo measures of cancer growth and metastasis.

### SUMMARY OF INVENTION

Cancer progression depends on an accumulation of metastasis-supporting physiological changes, which are regulated by cell-signaling molecules. In this regard, a disintegrin and metalloproteinase 8 (Adam8) is a transmembrane glycoprotein that is selectively expressed and induced by a variety of inflammatory stimuli. The inventors identified Adam8 as a sox2-dependent protein expressed in MDA-MB-231 breast cancer cells when cocultured with mesenchymal-stem-cell-derived myofibroblast-like cancer-associated fibroblasts (myCAF). The inventors previously found that myCAF-induced cancer stemness is required for the maintenance of the myCAF phenotype, suggesting that the initiation and maintenance of the myCAF phenotype require distinct cell-signaling crosstalk pathways between cancer cells and myCAF. Adam8 was identified as a candidate secreted protein induced by myCAF-mediated cancer stemness. Adam8 has a known sheddase function against which the inventors have developed an RNA aptamer, namely, Adam8-Apt1-26nt. The Adam8-Apt1-26nt-mediated blockade of the extracellular soluble Adam8 metalloproteinase domain abolishes the previously initiated myCAF phenotype, or, termed differently, blocks the maintenance of the myCAF phenotype. Consequently, cancer stemness is significantly decreased. Xenograft models show that Adam8-Apt-1-26nt administration is associated with decreased tumor growth and metastasis, while flow cytometric analyses demonstrate a significantly decreased fraction of myCAF after Adam8-Apt-1-26nt treatment. The role of soluble Adam8 in the maintenance of the myCAF phenotype has not been previously characterized and the results obtained by the inventors suggest that the signal pathways for the induction or initiation of the myCAF phenotype may be distinct from those involved with the maintenance of the myCAF phenotype.

In an embodiment, a composition is presented comprising: an RNA aptamer having at least 90% homology to SEQ ID NO:1, SEQ ID NO:2, SEQ ID NO:3, SEQ ID NO:4, SEQ ID NO:5, SEQ ID NO:6, SEQ ID NO:7, SEQ ID NO:8, or SEQ ID NO:9; and a pharmaceutically acceptable carrier. In some embodiments, the RNA aptamer is Apt-1 having SEQ ID NO:1. In other embodiments, the RNA aptamer is Apt-1-26nt having SEQ ID NO:7.

In another embodiment, a nucleic acid molecule not more than 80 nucleotides in length is presented comprising: an RNA aptamer having at least 90% homology to SEQ ID NO:1, SEQ ID NO:2, SEQ ID NO:3, SEQ ID NO:4, SEQ ID NO:5, SEQ ID NO:6, SEQ ID NO:7, SEQ ID NO:8, or SEQ ID NO:9. In some embodiments, the nucleic acid is Apt-1 having SEQ ID NO:1. In other embodiments, the nucleic acid is Apt-1-26nt having SEQ ID NO:7.

In a further embodiment, a method of treating a disease characterized by upregulated Adam8 in a patient in need thereof is presented comprising: administering to the patient in need thereof a therapeutically effective amount of a therapeutic agent comprising an RNA aptamer having a sequence of SEQ ID NO:1, SEQ ID NO:6, SEQ ID NO:7, SEQ ID NO:8, or SEQ ID NO:9.

The disease characterized by upregulated Adam8 may be selected from the group consisting of inflammatory diseases of the lung, inflammatory diseases of the central nervous system, inflammatory diseases of the bones and joints, inflammatory diseases of the circulatory system, asthma, atherosclerosis, liver injury and cancer. The RNA aptamer may bind to a soluble extracellular metalloproteinase domain of Adam8. In some embodiments, the disease associated with upregulated Adam8 expression may be a cancer. In some embodiments, the cancer may be selected from the group consisting of breast cancers, liver cancers, pancreatic cancers, brain cancers, colon cancers, renal cancers, bone cancers, lung cancers, and head and neck cancer. In an embodiment, the cancer is breast cancer and the therapeutic agent administered to the patient is the RNA aptamer having the sequence of SEQ ID NO:1 or SEQ ID NO:7. In another embodiment, the cancer is liver cancer and the therapeutic agent administered to the patient is the RNA aptamer having the sequence of SEQ ID NO:1 or SEQ ID NO:7.

In a further embodiment, a method of inhibiting growth of cancer cells in a patient in need thereof is presented comprising: administering to the patient in need thereof a therapeutically effective amount of a therapeutic agent comprising an RNA aptamer having a sequence of SEQ ID NO:1, SEQ ID NO:6, SEQ ID NO:7, SEQ ID NO:8, or SEQ ID NO:9, wherein the cancer cells are from a cancer characterized by upregulated Adam8 and wherein administration of the therapeutic agent inhibits the growth of the cancer cells in the patient. The cancer may be selected from the group consisting of breast cancers, liver cancers, pancreatic cancers, brain cancers, colon cancers, renal cancers, bone cancers, lung cancers, and head and neck cancers. In an embodiment, the cancer may be breast cancer and the therapeutic agent administered to the patient may be the RNA aptamer having the sequence of SEQ ID NO:1 or SEQ ID NO:7. In another embodiment, the cancer may be liver cancer and the therapeutic agent administered to the patient may be the RNA aptamer having the sequence of SEQ ID NO:1 or SEQ ID NO:7.

In a further embodiment, a kit for treating a disease characterized by upregulated Adam8 is presented comprising: a composition comprising an RNA aptamer having a sequence of SEQ ID NO:1, SEQ ID NO:6, SEQ ID NO:7, SEQ ID NO:8, or SEQ ID NO:9 and a pharmaceutically acceptable carrier, and instructions for use of the composition. In some embodiments, the disease characterized by upregulated Adam8 may be a cancer selected from the group consisting of breast cancers, liver cancers, pancreatic cancers, brain cancers, colon cancers, renal cancers, bone cancers, lung cancers, and head and neck cancers. In some embodiments, the RNA aptamer may be Apt-1 having SEQ ID NO:1 or Apt-1-26nt having SEQ ID NO:7.

In a further embodiment, a method of reversing a myofibroblast cancer-associated fibroblast (myCAF) phenotype in a patient in need thereof is presented comprising: diagnosing or having diagnosed the patient with a cancer characterized by increased expression of Adam8 as compared to a control; determining or having determined presence of the myCAF phenotype in the patient; and administering to the patient in need thereof a therapeutically effective amount of a therapeutic agent comprising an RNA aptamer having a sequence of SEQ ID NO:1, SEQ ID NO:6, SEQ ID NO:7, SEQ ID NO:8, or SEQ ID NO:9 wherein the administration of the therapeutic agent reverses the myCAF phenotype in the patient. The cancer may be selected from the group consisting of breast cancers, liver cancers, pancreatic cancers, brain cancers, colon cancers, renal cancers, bone cancers, lung cancers, and head and neck cancer. The therapeutic agent administered to the patient may be the RNA aptamer having the sequence of SEQ ID NO:1 or SEQ ID NO:7. In an embodiment, the cancer may be breast cancer and the therapeutic agent administered to the patient may be the RNA aptamer having the sequence of SEQ ID NO:1 or SEQ ID NO:7. In another embodiment, the cancer may be liver cancer and the therapeutic agent administered to the patient may be the RNA aptamer having the sequence of SEQ ID NO:1 or SEQ ID NO:7.

The myCAF phenotype may be characterized or determined by the presence of an increased expression level of at least one of alpha-smooth muscle actin (α-SMA), tenascin C (TenC), vimentin A (Vim A) or a combination thereof as compared to a control. The administration of the therapeutic agent may decrease the expression level of the at least one of α-SMA, TenC, Vim A or the combination thereof to reverse the myCAF phenotype.

In a further embodiment, a method of inhibiting cancer cell metastasis in a patient in need thereof is presented comprising: diagnosing or having diagnosed the patient with a cancer characterized by increased expression of Adam8 as compared to a control and administering to the patient in need thereof a therapeutically effective amount of a therapeutic agent comprising an RNA aptamer having a sequence of SEQ ID NO:1, SEQ ID NO:6, SEQ ID NO:7, SEQ ID NO:8, or SEQ ID NO:9, wherein the administration of the therapeutic agent inhibits cancer cell metastasis in the patient. The cancer may be selected from the group consisting of breast cancers, liver cancers, pancreatic cancers, brain cancers, colon cancers, renal cancers, bone cancers, lung cancers, and head and neck cancers. The therapeutic agent administered to the patient may be the RNA aptamer having the sequence of SEQ ID NO:1 or SEQ ID NO:7. In an embodiment, the cancer may be breast cancer and the therapeutic agent administered to the patient may be the RNA aptamer having the sequence of SEQ ID NO:1 or SEQ ID NO:7. In another embodiment, the cancer may be liver cancer and the therapeutic agent administered to the patient may be the RNA aptamer having the sequence of SEQ ID NO:1 or SEQ ID NO:7.

In a further embodiment, a method of decreasing cancer cell stemness in a patient in need thereof is presented comprising: diagnosing or having diagnosed the patient with a cancer characterized by increased expression of Adam8 as compared to a control and administering to the patient in need thereof a therapeutically effective amount of a therapeutic agent comprising an RNA aptamer having a sequence of SEQ ID NO:1, SEQ ID NO:6, SEQ ID NO:7, SEQ ID NO:8, or SEQ ID NO:9, wherein the administration of the therapeutic agent decreases cancer cell stemness in the patient. The cancer may be selected from the group consisting of breast cancers, liver cancers, pancreatic cancers, brain cancers, colon cancers, renal cancers, bone cancers, lung cancers, and head and neck cancers. The therapeutic agent administered to the patient may be the RNA aptamer having the sequence of SEQ ID NO:1 or SEQ ID NO:7. In an embodiment, the cancer may be breast cancer and the therapeutic agent administered to the patient may be the RNA aptamer having the sequence of SEQ ID NO:1 or SEQ ID NO:7. In another embodiment, the cancer may be liver cancer and the therapeutic agent administered to the patient may be the RNA aptamer having the sequence of SEQ ID NO:1 or SEQ ID NO:7.

In a further embodiment, a method of substantially silencing a gene of interest in a patient in need thereof is presented comprising: identifying or having identified increased expression of the gene of interest as compared to a control wherein the gene of interest is Adam8 and administering to the patient a therapeutically effective amount of a therapeutic agent comprising an RNA aptamer having a sequence of SEQ ID NO:1, SEQ ID NO:6, SEQ ID NO:7, SEQ ID NO:8, or SEQ ID NO:9, wherein the therapeutic agent binds to at least a portion of the gene of interest to silence the gene of interest. The therapeutic agent may bind to a soluble extracellular metalloproteinase domain of the Adam8 gene. The therapeutic agent administered to the patient may be the RNA aptamer having the sequence of SEQ ID NO:1 or SEQ ID NO:7.

### BRIEF DESCRIPTION OF THE DRAWINGS

For a fuller understanding of the invention, reference should be made to the following detailed description, taken in connection with the accompanying drawings, in which:

FIG. 1 is an image depicting Adam8 Apt-1-26nt aptamer does not induce immunogenicity.

FIG. 2 is a table of RNA seq data showing a common gene list.

FIG. 3A-B are a series of Western blots depicting CA12 and CDH6 knockdown in MDA-MB-231 cells, (A) CA12 protein and (B) CDH6 protein.

FIG. 3C-D are a series of graphs depicting SMA/TNC/VIM gene expression quantified with RT-PCR in (C) MDA-MB-231-CA12-KD or (D) CDH6-KD co-cultured cells.

FIG. 4 is a graph depicting α-SMA gene expression in MSC cells, co-cultured with MDA-MB-231 cells or MDA-MB-231 cells transfected with Adam8 shRNA lentiviral particles or control shRNA lentiviral particles at different time points.

FIG. 5 is a listing of the Adam8 RNA aptamer sequences.

FIG. 6A is a series of graphs depicting the synthesis and characterization of aptamer Apt-1 targeting Adam8. (A) Apt-1 binding affinity and specificity, in vivo and in vitro half-life, and intravenous and subcutaneous administration kinetics.

FIG. 6B is an image depicting the synthesis and characterization of aptamer Apt-1 targeting Adam8. (B) the structures of the candidate aptamers (Apt-1 through 5) targeting Adam8 sequences and theoretic tertiary structure.

FIG. 6C is a series of images depicting the synthesis and characterization of aptamer Apt-1 targeting Adam8. (C) the structures and sequences of the Apt-1 deletion mutant constructs (Mut1 through 4).

FIG. 6D is a graph depicting the synthesis and characterization of aptamer Apt-1 targeting Adam8. (D) the Apt-1 deletion mutant activities (n=3).

FIG. 6E is a graph depicting the synthesis and characterization of aptamer Apt-1 targeting Adam8. (E) Apt-1 cross-reactivity with Adam10 and Adam 17 (n=3).

FIG. 6F is a series of images depicting the synthesis and characterization of aptamer Apt-1 targeting Adam8. (F) Apt-1 is extracellular in the presence of MDA-MB-231 and HepG2 cells (20×20).

FIG. 7A is a series of graphs depicting the effect of Adam8 and cancer stemness on the myCAF phenotype. (A) Adam8 ablation effect on time-dependent myCAF expression of α-SMA in MDA-MB231+MSC and HepG2+MSC co-cultures (n=3).

FIG. 7B is a series of graphs depicting the effect of Adam8 and cancer stemness on the myCAF phenotype. (B) Effect of Adam8 ablation on time-dependent cancer cell expression of sox2 in MDA-MB231+MSC and HepG2+MSC co-cultures (n=3).

FIG. 7C is a series of graphs depicting the effect of Adam8 and cancer stemness on the myCAF phenotype. (C) Effect of Adam8 ablation on time-dependent cancer cell expression of Oct4 in MDA-MB231+MSC and HepG2+MSC co-cultures (n=3).

FIG. 7D is a series of graphs depicting the effect of Adam8 and cancer stemness on the myCAF phenotype. (D) Conditioned media studies examining myCAF marker expression and effect of cancer+MSC (and cancer (sox2-KD)+MSC) coculture media for which Adam8 has been depleted (n=3).

FIG. 7E is a series of graphs depicting the effect of Adam8 and cancer stemness on the myCAF phenotype. (E) Cancer stemness markers (sox2, Nanog, and Oct4) in MDA-MB-231 and HepG2 cocultures with MSC, Apt-1, and/or the stemness inhibitor BBI 608. The uncropped blots are shown in FIG. 12.

FIG. 8A is a series of graphs depicting the time-dependent effect of Apt-1 on myCAF and cancer stemness markers. (A) Time-dependent effect of Apt-1 on myCAF and cancer stemness markers in MDA-MB-231+MSC cocultures (n=3).

FIG. 8B is a series of graphs depicting the time-dependent effect of Apt-1 on myCAF and cancer stemness markers. (B) Time-dependent effect of Apt-1 on myCAF and cancer stemness markers in HepG2+MSC cocultures (n=3).

FIG. 8C is a series of images depicting the time-dependent effect of Apt-1 on myCAF and cancer stemness markers. (C) Tumorsphere assay for MDA-MB-231 and HepG2 cocultures with MSC and Apt-1 (10×20).

FIG. 8D is a graph depicting the time-dependent effect of Apt-1 on myCAF and cancer stemness markers. (D) Apt-1 pulldown studies performed based on binding competition with 200-fold excess non-labeled Apt-1.

FIG. 9 is a series of images depicting results of the MDA-MB-231 cell invasion assay (16 h).

FIG. 10 is a series of images depicting the results of the MDA-MB-231 cell wound healing assay.

FIG. 11A is a series of images depicting MDA-MB-231+MSC xenografts in NOD-scid mice. (A) Time course of MDA-MB-231 luciferase activity in saline- and Apt-1-26nt-treated mice.

FIG. 11B is a series of images depicting MDA-MB-231+MSC xenografts in NOD-scid mice. (B) Time course of MDA-MB-231 luminescence activity in saline- and Apt-1-26nt-treated mice (n=3).

FIG. 11C-D are a series of images depicting MDA-MB-231+MSC xenografts in NOD-scid mice. (C) Breast, lung, and liver MDA-MB-231 luciferase activity in saline- and Apt-1-26nt-treated mice after sacrifice at week 6. (D) Flow cytometry regarding RFP-MDA-MB-231, GFP-MSC, and α-SMA inducible BFP-myCAF in saline- and Apt-1-26nt-treated mice after sacrifice at week 6. ** and * indicate t-test p-values<0.05.

FIG. 12 is a graph depicting a Western blot showing Adam8 knockdown in MDA-MB-231 cells.

### DETAILED DESCRIPTION OF THE PREFERRED EMBODIMENT

In the following detailed description of the preferred embodiments, reference is made to the accompanying drawings, which form a part hereof, and within which are shown by way of illustration specific embodiments by which the invention may be practiced. It is to be understood that other embodiments may be utilized, and structural changes may be made without departing from the scope of the invention.

### Abbreviations

α-SMA—alpha smooth muscle actin;
TenC—Tenascin C;
Vim—Vimentin;
sox2—SRY related HMG box transcription factor 2;
Oct4—Octamer binding transcription factor 4;
Nanog—Homeobox protein Nanog;
CA12—carbonic anhydrase 12;
CDH6—Cadherin 6;
Adam8-KD—Adam8 shRNA knockdown
Sox2-KD—Sox2 shRNA knockdown

Apt-1 -
5′-UCUGCACGUUCGAAUAAGUCUCCGGUGUUUCGAGACCCUU-3′
Apt1-1-26nt (or Mut 2) -
5′-UAAGUCUCCGGUGUUUCGAGACCCUU-3′

### Definitions

Unless otherwise defined, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are described herein. All publications mentioned herein are incorporated herein by reference in their entirety to disclose and describe the methods and/or materials in connection with which the publications are cited. It is understood that the present disclosure supercedes any disclosure of an incorporated publication to the extent there is a contradiction.

As used herein, the singular forms “a,” “an” and “the” include plural referents unless the context clearly dictates otherwise.

As used in this specification and the appended claims, the term “or” is generally employed in its sense including “and/or” unless the context clearly dictates otherwise.

All numerical designations, such as pH, temperature, time, concentration, and molecular weight, including ranges, are approximations which are varied up or down by increments of 1.0, 0.1, 0.01 or 0.001 as appropriate. It is to be understood, even if it is not always explicitly stated that all numerical designations are preceded by the term “about”. It is also to be understood, even if it is not always explicitly stated, that the reagents described herein are merely exemplary and that equivalents of such are known in the art and can be substituted for the reagents explicitly stated herein.

Concentrations, amounts, solubilities, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include the individual values and sub-ranges within the indicated range. Thus, included in this numerical range are individual values such as 2, 3, and 4 and sub-ranges such as from 1-3, from 2-4 and from 3-5, etc. This same principle applies to ranges reciting only one numerical value. Furthermore, such an interpretation should apply regardless of the range or the characteristics being described.

As used herein, the term “comprising” is intended to mean that the products, compositions, and methods include the referenced components or steps, but not excluding others. “Consisting essentially of” when used to define products, compositions, and methods, shall mean excluding other components or steps of any essential significance that affect the novel characteristics of the invention as described herein. Thus, a composition consisting essentially of the recited components would not exclude trace contaminants and pharmaceutically acceptable carriers. “Consisting of” shall mean excluding more than trace elements of other components or steps.

As used herein, “about” means approximately or nearly and in the context of a numerical value or range set forth means ±10% of the numerical.

As used herein “patient” is used to describe a mammal, preferably a human, to whom treatment is administered, including prophylactic treatment with the compositions of the present invention. Non-limiting examples of mammals include humans, rodents, aquatic mammals, domestic animals such as dogs and cats, farm animals such as sheep, pigs, cows and horses. “Patient” and “subject” are used interchangeably herein.

“Administering” or “administration” as used herein refers to the process by which the compositions of the present invention are delivered to the patient. The compositions may be administered in various ways, including but not limited to, orally, nasally, subcutaneously, and parenterally.

“Parenteral administration” as used herein refers to modes of administration other than enteral and topical administration, usually by injection, and includes, but is not limited to, intravenous, intramuscular, intraarterial, intrathecal, intracapsular, intraorbital, intracardiac, intradermal, intraperitoneal, transtracheal, subcutaneous, subcuticular, intra-articular, subcapsular, intrathecal, intraventricular, intracisternal, intranigral, subarachnoid, intraspinal, and intrasternal injection and infusion. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

A “therapeutic agent” as used herein refers to a substance, composition, compound, chemical, component or extract that has measurable specified or selective physiological activity when administered to an individual in a therapeutically effective amount. In some embodiments, the therapeutic agent may be a an aptamer targeting a gene of interest. Examples of therapeutic agents as used in the present invention include, but are not limited to, RNA aptamers. At least one therapeutic agent is used in the compositions of the present invention, however in some embodiments, multiple therapeutic agents are used. In some embodiments, the novel RNA aptamers described herein may be combined with another therapeutic agent that targets a different area of the gene or targets a different disease target. In some embodiments, one or more therapeutic agents may be encapsulated within a nanoparticle. In some embodiments, the therapeutic agent is used to treat a disease characterized by upregulated Adam8. Examples of such diseases include, but are not limited to, inflammatory diseases of the lung, inflammatory diseases of the central nervous system, inflammatory diseases of the bones and joints, inflammatory diseases of the circulatory system, atherosclerosis, liver injury, asthma, and cancers such as breast cancers, liver cancers, pancreatic cancers, brain cancers, colon cancers, renal cancers, bone cancers, lung cancers, and head and neck cancers.

The terms “reduce or inhibit” as used herein refers to the ability to cause an overall decrease of 10%, 20%, 30%, 40%, 50%, 60%, 70%, 75%, 80%, 85%, 90%, 95%, or greater. Reduce or inhibit can refer, for example, to the symptoms of the disorder being treated, the presence or size of metastases (in the case of cancer), or the size of the primary tumor (in the case of cancer).

A “therapeutically effective amount” as used herein is defined as concentrations or amounts of components which are sufficient to effect beneficial or desired clinical results, including, but not limited to, any one or more of treating symptoms of a disease characterized by upregulated Adam8, such as cancer, and preventing a disease characterized by upregulated Adam8, particularly a cancer characterized by upregulated Adam8. Compositions of the present invention can be used to effect a favorable change in the condition whether that change is an improvement, such as stopping, reversing, or a complete elimination of symptoms due to the disease. In some instances of cancer, the favorable change may be reducing growth or metastasis of cancer cells, apoptosis or otherwise killing of cancer cells. In accordance with the present invention, a suitable single dose size is a dose that is capable of preventing or alleviating (reducing or eliminating) a symptom in a patient when administered one or more times over a suitable time period. One of skill in the art can readily determine appropriate single dose sizes for systemic administration based on the size of the animal and the route of administration. The dose may be adjusted according to response.

The dosing of compounds and compositions to obtain a therapeutic or prophylactic effect is determined by the circumstances of the patient, as is known in the art. The dosing of a patient herein may be accomplished through individual or unit doses of the compounds or compositions herein or by a combined or prepackaged or pre-formulated dose of a compounds or compositions.

The amount of the compound in the drug composition will depend on absorption, distribution, metabolism, and excretion rates of the drug as well as other factors known to those of skill in the art. Dosage values may also vary with the severity of the condition to be alleviated. The compounds may be administered once, or may be divided and administered over intervals of time. It is to be understood that administration may be adjusted according to individual need and professional judgment of a person administrating or supervising the administration of the compounds used in the present invention.

The dose of the compounds administered to a subject may vary with the particular composition, the method of administration, and the particular disorder being treated. The dose should be sufficient to affect a desirable response, such as a therapeutic or prophylactic response against a particular disorder or condition. It is contemplated that one of ordinary skill in the art can determine and administer the appropriate dosage of compounds disclosed in the current invention according to the foregoing considerations.

Dosing frequency for the composition includes, but is not limited to, at least about once every three weeks, once every two weeks, once a week, twice a week, three times a week, four times a week, five times a week, six times a week, or daily. In some embodiments, the interval between each administration is less than about a week, such as less than about any of 6, 5, 4, 3, 2, or 1 day. In some embodiments, the interval between each administration is constant. For example, the administration can be carried out daily, every two days, every three days, every four days, every five days, or weekly. In some embodiments, the administration can be carried out twice daily, three times daily, or more frequently. Administration can also be continuous and adjusted to maintaining a level of the compound within any desired and specified range.

The administration of the composition can be extended over an extended period of time, such as from about a week or shorter up to about a year or longer. For example, the dosing regimen can be extended over a period of any of about 2, 3, 4, 5, 6, 7, 8, 9, 10, 11, and 12 months. In some embodiments, there is no break in the dosing schedule. In some embodiments, the interval between each administration is no more than about a week.

The therapeutic agents used in the present invention may be administered individually, or in combination with or concurrently with one or more other therapeutic agents used against diseases characterized by upregulated Adam8, including cancers characterized by upregulated Adam8. Additionally, in the case of cancers characterized by upregulated Adam8, therapeutic agents used in the present invention may be administered in combination with or concurrently with other therapeutics for cancers such as immunomodulatory compounds and chemotherapeutics.

“Prevention” or “preventing” or “prophylactic treatment” as used herein refers to any of: halting the effects of diseases characterized by upregulated Adam8, reducing the effects of diseases characterized by upregulated Adam8, reducing the incidence of diseases characterized by upregulated Adam8, reducing the development of diseases characterized by upregulated Adam8, delaying the onset of symptoms of diseases characterized by upregulated Adam8, increasing the time to onset of symptoms of diseases characterized by upregulated Adam8, and reducing the risk of development of diseases characterized by upregulated Adam8. In some embodiments, the disease characterized by upregulated Adam8 is a cancer characterized by upregulated Adam8. In some embodiments, prevention is shown by decreasing or inhibiting metastasis of cancer cells.

“Inhibition of metastasis” as used herein refers to inhibition of the spread of cancer cells to a different part of the body from the location of the primary tumor.

“Cancer” “tumor”, “cancerous”, and malignant” as used herein, refer to the physiological condition in mammals that is typically characterized by unregulated cell growth. Examples of cancers benefited by the present invention include, but are not limited to, solid tumors, in particular those characterized by or exhibiting upregulated/overexpression of Adam8 as compared to a normal control.

Cancers capable of being treated with the therapeutic agent described herein include, but are not limited to: breast cancers including, but not limited to, ductal carcinoma in situ, Paget's disease of the breast, lobular carcinoma in situ, mucinous neoplasm, medullary carcinoma, inflammatory breast cancer, metaplastic carcinoma, triple-negative breast cancer, metastatic breast cancer, male breast cancer, ductal carcinoma, invasive lobular carcinoma, Phyllodes tumor, angiosarcoma, HER-2 positive breast cancer, HER2-negative breast cancer, HER2-low breast cancer, hormone-receptor positive breast cancers such as estrogen receptor positive and progesterone receptor positive breast cancers, estrogen-negative breast cancer, progesterone negative breast cancer, breast sarcoma; liver cancers including, but not limited to, hepatocellular carcinoma, cholangiocarcinoma, angiosarcoma, hemangiosarcoma; pancreatic cancers, including, but not limited to, exocrine pancreatic cancer such as adenocarcinoma and neuroendocrine pancreatic cancer; brain cancers including, but not limited to, meningioma, glioblastoma multiforme, oligodendroglioma, brainstem glioma, anaplastic astrocytoma, primitive neuroectodermal tumor, chordoma, germinoma, astrocytomas, medulloblastoma, craniopharyngioma, schwannoma, primary central nervous system lymphoma, pilocytic astrocytoma, optic nerve glioma, pineoblastoma, gliomas, ependymoma, pituitary adenoma, vestibular schwannoma, germ cell tumor, mixed glioma, diffuse astrocytomas, choroid plexus papilloma; colon cancers including, but not limited to, adenocarcinomas, primary colorectal lymphomas, gastrointestinal stromal tumors, leiomyosarcomas, carcinoid tumors; renal cancers including, but not limited to, renal cell cancer, transitional cell carcinoma, renal oncocytoma, collecting duct carcinoma, Wilms' tumor, sarcoma, renal medullary carcinoma, sarcomatoid carcinoma, clear-cell renal-cell carcinoma, papillary renal cell carcinoma, chromophobe renal cell carcinoma, chromophobe; bone cancers including, but not limited to, osteosarcoma, Ewing tumor, chondrosarcoma, high-grade undifferentiated pleomorphic sarcoma, fibrosarcoma, malignant fibrous histiocytoma, Giant cell tumor, chordoma, multiple myeloma, non-Hodgkin lymphoma of bone; lung cancers including, but not limited to, non-small cell lung cancer, large cell carcinoma, salivary gland-like carcinoma of the lung, small-cell carcinoma, squamous cell carcinoma, adenosquamous lung carcinoma, adenocarcinoma, mesothelioma, large cell neuroendocrine carcinoma; and head and neck cancers (such as cancers in the larynx, throat, lips, mouth, nose and salivary glands) including, but not limited to, hypopharyngeal cancer, laryngeal cancer, lip and oral cavity cancer, metastatic squamous neck cancer with occult primary, nasopharyngeal cancer, oropharyngeal cancer, paranasal sinus and nasal cavity cancer, salivary gland cancer.

“Treatment” or “treating” as used herein refers to any of the alleviation, amelioration, elimination and/or stabilization of a symptom, as well as delay in progression of a symptom of a particular disease or disorder, particularly those diseases characterized by upregulated Adam8. For example, “treatment” of cancer characterized by upregulated Adam8 may include any one or more of the following: amelioration and/or elimination of one or more symptoms associated with cancer, reduction of one or more symptoms of cancer, stabilization of symptoms of cancer, and delay in progression of one or more symptoms of cancer.

A cancer is “responsive” to a therapeutic agent or there is a “good response” to a treatment if its rate of growth is inhibited as a result of contact with the therapeutic agent, compared to its growth in the absence of contact with the therapeutic agent, if metastasis is inhibited, if the cancer cells exhibit apoptosis or otherwise are killed, etc. Growth of a cancer can be measured in a variety of ways, for instance, the characteristic, e.g., size of a tumor or the expression of tumor markers appropriate for that tumor type may be measured.

A cancer is “non-responsive” or has a “poor response” to a therapeutic agent or there is a poor response to a treatment if its rate of growth is not inhibited, or inhibited to a very low degree, as a result of contact with the therapeutic agent when compared to its growth in the absence of contact with the therapeutic agent, if metastasis occurs, etc. As stated above, growth of a cancer can be measured in a variety of ways, for instance, the size of a tumor or the expression of tumor markers appropriate for that tumor type may be measured.

The pharmaceutical compositions of the instant invention may comprise sufficient genetic material to produce a therapeutically effective amount of the aptamer of interest, i.e., an amount sufficient to reduce or ameliorate symptoms of the disease characterized by upregulated Adam8, such as cancer, or an amount sufficient to confer the desired benefit. The pharmaceutical compositions of the subject invention can be formulated according to known methods for preparing pharmaceutically useful compositions. When the therapeutic agents of the invention are prepared for administration, they are preferably combined with a pharmaceutically acceptable carrier, diluent or excipient to form a pharmaceutical formulation, or unit dosage form. The total active ingredients in such formulations include from 0.1 to 99.9% by weight of the formulation. Furthermore, as used herein, the phrase “pharmaceutically acceptable carrier” means any of the standard pharmaceutically acceptable carriers. The pharmaceutically acceptable carrier can include excipients, diluents, adjuvants, and vehicles, as well as implant carriers, and inert, non-toxic solid or liquid fillers, diluents, or encapsulating material that does not react with the active ingredients of the invention and do not themselves induce the production of antibodies harmful to the individual receiving the composition, and which may be administered without undue toxicity. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. Example of suitable excipients include, but are not limited to, sorbitol, Tween80, and liquids such as water, saline, glycerol, and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. Formulations are described in a number of sources that are well known and readily available to those skilled in the art. For example, Remington's Pharmaceutical Sciences (Martin E W [1995] Easton Pennsylvania, Mack Publishing Company, 19th ed.) describes formulations which can be used in connection with the subject invention.

For ease of administration, the subject compounds may be formulated into various pharmaceutical forms. As appropriate compositions there may be cited all compositions usually employed for systemically or topically administering drugs. To prepare the pharmaceutical compositions of this invention, a therapeutically effective amount of RNA aptamer, as the active ingredient is combined in intimate admixture with a pharmaceutically acceptable carrier, which may take a wide variety of forms depending on the form of preparation desired for administration. These pharmaceutical compositions are desirably in unitary dosage form suitable, preferably, for administration nasally, orally, percutaneously, subcutaneously, or by parenteral injection. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules and tablets. Because of their ease in administration, tablets and capsules often represent the most advantageous oral dosage unit form, in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution.

The terms “overexpression”, “increased expression”, or “upregulated” and “underexpression”, “decreased expression”, or “downregulated” as used herein refers to the expression of a gene of a patient at a greater or lesser level, respectively, than the normal or control expression of the gene, as measured by gene expression product expression, such as mRNA or protein expression, in a sample that is greater than the standard of error of the assay used to assess the expression.

The term “baseline level” or “control level” of biomarker expression or activity refers to the level against which biomarker expression in the test sample can be compared. In some embodiments, the baseline level can be a normal level, meaning the level in a sample from a normal patient. This allows a determination based on the baseline level of biomarker expression or biological activity, whether a sample to be evaluated for disease cell growth has a measurable increase, decrease, or substantially no change in biomarker expression as compared to the baseline level. The term “negative control” used in reference to a baseline level of biomarker expression generally refers to a baseline level established in a sample from the subject or from a population of individuals which is believed to be normal (e.g. non-tumorous, not undergoing neoplastic transformation, not exhibiting inappropriate cell growth). In other embodiments, the baseline level can be indicative of a positive diagnosis of disease (e.g. positive control). The term “positive control” as used herein refers to a level of biomarker expression or biological activity established in a sample from a subject, from another individual, or from a population of individuals, where the sample was believed, based on data from that sample, to have the disease (e.g. tumorous, cancerous, exhibiting inappropriate cell growth). In other embodiments, the baseline level can be established from a previous sample from the subject being tested, so that the disease progression or regression of the subject can be monitored over time and/or the efficacy of treatment can be evaluated.

The term “biomarker” is used herein to refer to a molecule whose level of nucleic acid or protein product has a quantitatively differential concentration or level with respect to an aspect of a biological state of a subject. “Biomarker” is used interchangeably with “marker” herein. The level of the biomarker can be measured at both the nucleic acid level as well as the polypeptide level. At the nucleic acid level, a nucleic acid gene or a transcript which is transcribed from any part of the subject's chromosomal and extrachromosomal genome, including for example the mitochondrial genome, may be measured. Preferably an RNA transcript, more preferably an RNA transcript includes a primary transcript, a spliced transcript, an alternatively spliced transcript, or an mRNA of the biomarker is measured. At the polypeptide level, a pre-propeptide, a propeptide, a mature peptide or a secreted peptide of the biomarker may be measured. In some embodiments, the Adam8 gene products are used as biomarkers.

The present invention provides a method of substantially silencing a target gene of interest or targeted allele for the gene of interest in order to provide a therapeutic effect. Use of this strategy results in markedly diminished in vitro and in vivo expression of the targeted gene(s) and is useful in reducing expression of the targeted gene(s) in order to provide therapy for human diseases, such as treatment of diseases characterized by upregulated Adam8. As used herein the term “substantially silencing” or “substantially silenced” refers to decreasing, reducing, or inhibiting the expression of the target gene or target allele by at least about 5%, 10%, 15%, 20%, 25%, 30%, 35%, 40%, 45%, 50%, 55%, 60%, 65%, 70%, 75%, 80%, 85%, 90%, 95%, to 100%. As used herein the term “therapeutic effect” refers to a change in the associated abnormalities of the disease state, including pathological and behavioral deficits; a change in the time to progression of the disease state; a reduction, lessening, or alteration of a symptom of the disease; or an improvement in the quality of life of the person afflicted with the disease. Therapeutic effect can be measured quantitatively by a physician or qualitatively by a patient afflicted with the disease state targeted by the aptamer. In certain embodiments wherein both the mutant and wild type allele are substantially silenced, the term therapeutic effect defines a condition in which silencing of the wild type allele's expression does not have a deleterious or harmful effect on normal functions such that the patient would not have a therapeutic effect. In some embodiments, the target gene or gene of interest is Adam8.

The term “aptamer” as used herein refers to single stranded oligonucleotides that can naturally fold into different 3-dimensional structures, which have the capability of binding specifically to biosurfaces, a target molecule or compound, or a moiety. The term “conformational change” refers to the process by which a nucleic acid, such as an aptamer, adopts a different secondary or tertiary structure. The term “fold” may be substituted for conformational change. Aptamers are typically oligonucleotides that may be single stranded oligodeoxynucleotides, oligoribonucleotides, or modified oligodeoxynucleotide or oligoribonucleotides. The term “modified” encompasses nucleotides with a covalently modified base and/or sugar. For example, modified nucleotides include nucleotides having sugars which are covalently attached to low molecular weight organic groups other than a hydroxyl group at the 3′ position and other than a phosphate group at the 5′ position. Thus modified nucleotides may also include 2′ substituted sugars such as 2′-O-methyl-; 2-O-alkyl; 2-O-allyl; 2′-S-alkyl; 2′-S-allyl; 2′-fluoro-; 2′-halo or 2-azido-ribose, carbocyclic sugar analogues a-anomeric sugars; epimeric sugars such as arabinose, xyloses or lyxoses, pyranose sugars, furanose sugars, and sedoheptulose. Modified nucleotides are known in the art and include, by example and not by way of limitation, alkylated purines and/or pyrimidines; acylated purines and/or pyrimidines; or other heterocycles. In some embodiments, the aptamers used herein target Adam8, particularly the extracellular Adam8 soluble metalloproteinase domain.

As used herein, a “target” or “target molecule” refers to a biomolecule that could be the focus of a therapeutic drug strategy or diagnostic assay, including, without limitation, proteins or portions thereof, enzymes, peptides, enzyme inhibitors, hormones, carbohydrates, glycoproteins, lipids, phospholipids, nucleic acids, and generally, any biomolecule capable of turning a biochemical pathway on or off or modulating it, or which is involved in a predictable biological response. Targets can be free in solution, like thrombin, or associated with cells or viruses, as in receptors or envelope proteins. Any ligand that is of sufficient size to be specifically recognized by an oligonucleotide sequence can be used as the target. Thus, glycoproteins, proteins, carbohydrates, membrane structures, receptors, organelles, and the like can be used as the complexation targets. A wide variety of materials can serve as targets. These materials include intracellular, extracellular, and cell surface proteins, peptides, glycoproteins, carbohydrates, including glycosaminoglycans, lipids, glycolipids and certain oligonucleotides.

The term “ligand” as used herein refers to a molecule or other chemical entity having a capacity for binding to a target. A ligand can comprise a peptide, an oligomer, a nucleic acid (e.g., an aptamer), a small molecule (e.g., a chemical compound), an antibody or fragment thereof, nucleic acid-protein fusion, and/or any other affinity agent. Thus, a ligand can come from any source, including libraries, particularly combinatorial libraries, such as the aptamer libraries disclosed herein, phage display libraries, or any other library as would be apparent to one of ordinary skill in the art after review of the disclosure of the present invention presented herein. In some embodiments, the ligand is a nucleic acid, more particularly an aptamer. In some embodiments, the aptamer is an RNA aptamer.

As used herein, the term “nucleic acid” and “polynucleotide” refers to deoxyribonucleotides or ribonucleotides and polymers thereof in either single- or double-stranded form, composed of monomers (nucleotides) containing a sugar, phosphate and a base that is either a purine or pyrimidine. Unless specifically limited, the term encompasses nucleic acids containing known analogs of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues.

A “nucleic acid fragment” is a portion of a given nucleic acid molecule. Deoxyribonucleic acid (DNA) in the majority of organisms is the genetic material while ribonucleic acid (RNA) is involved in the transfer of information contained within DNA into proteins. The term “nucleotide sequence” refers to a polymer of DNA or RNA which can be single- or double-stranded, optionally containing synthetic, non-natural or altered nucleotide bases capable of incorporation into DNA or RNA polymers.

The terms “nucleic acid,” “nucleic acid molecule,” “nucleic acid fragment,” “nucleic acid sequence or segment,” or “polynucleotide” may also be used interchangeably with gene, cDNA, DNA and RNA encoded by a gene, e.g., genomic DNA, and even synthetic DNA sequences. The term also includes sequences that include any of the known base analogs of DNA and RNA. Nucleic acids include one or more types of polydeoxyribonucleotides (containing 2-deoxy-D-ribose), polyribonucleotides (containing D-ribose), and any other type of polynucleotide that is an N-glycoside of a purine or pyrimidine base, or modified purine or pyrimidine bases (including abasic sites). The term “nucleic acid,” as used herein, also includes polymers of ribonucleosides or deoxyribonucleosides that are covalently bonded, typically by phosphodiester linkages between subunits, but in some cases by phosphorothioates, methylphosphonates, and the like. “Nucleic acids” include single- and double-stranded DNA, as well as single- and double-stranded RNA. Exemplary nucleic acids include, without limitation, aptamers, gDNA; hnRNA; mRNA; rRNA, tRNA, micro RNA (miRNA), small interfering RNA (siRNA), small nucleolar RNA (snORNA), small nuclear RNA (snRNA), and small temporal RNA (stRNA), and the like, and any combination thereof.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis that encode the native protein, as well as those that encode a polypeptide having amino acid substitutions. Generally, nucleotide sequence variants of the invention will have in at least 40%, 50%, 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence.

As used herein, “sequence identity” or “identity” or “homology” in the context of two nucleic acid sequences makes reference to a specified percentage of residues in the two sequences that are the same when aligned by sequence comparison algorithms or by visual inspection, i.e. the degree of complementarity between two or more polynucleotide or polypeptide sequences.

As used herein, “percentage of sequence identity” or “percentage of homology” means the value determined by comparing two optimally aligned sequences, wherein the portion of the polynucleotide sequence may comprise additions or deletions (i.e., gaps) as compared to the reference sequence (which does not comprise additions or deletions) for optimal alignment of the two sequences. The percentage is calculated by determining the number of positions at which the identical nucleic acid base occurs in both sequences to yield the number of matched positions, dividing the number of matched positions by the total number of positions in the window of comparison, and multiplying the result by 100 to yield the percentage of sequence identity.

The term “substantial identity” or “substantial homology” of polynucleotide sequences means that a polynucleotide comprises a sequence that has at least 70%, 71%, 72%, 73%, 74%, 75%, 76%, 77%, 78%, or 79%; at least 80%, 81%, 82%, 83%, 84%, 85%, 86%, 87%, 88%, or 89%; at least 90%, 91%, 92%, 93%, or 94%; or even at least 95%, 96%, 97%, 98%, or 99% sequence identity, compared to a reference sequence using one of the alignment programs described using standard parameters.

The expression vectors useful in the present invention are constructed using known techniques to at least provide as operatively linked components in the direction of transcription, control elements including a transcriptional initiation region, the DNA of interest and a transcriptional termination region. The control elements are selected to be functional in a mammalian cell. The resulting construct which contains the operatively linked components is flanked (5′ and 3′) with functional sequences, such as sequences encoding an aptamer.

The selected nucleotide sequence is operably linked to control elements that direct the transcription or expression thereof in the subject in vivo. Such control elements can comprise control sequences normally associated with the selected gene. Alternatively, heterologous control sequences can be employed. Useful heterologous control sequences generally include those derived from sequences encoding mammalian or viral genes. Examples include, but are not limited to, the SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), a Rous sarcoma virus (RSV) promoter, pol II promoters, pol III promoters, synthetic promoters, hybrid promoters, and the like. In addition, sequences derived from nonviral genes, such as the murine metallothionein gene, will also find use herein. Such promoter sequences are commercially available.

The term “promoter,” as used herein refers to a region or regions of a nucleic acid sequence that regulates transcription.

The inventors have characterized a novel RNA aptamer, Apt-1, directed against the extracellular Adam8 soluble metalloproteinase domain. In MSC co-culture studies employing MDA-MB-231 breast cancer cells and HepG2 liver cancer cells, Apt-1 inhibits MSCs' adoption of the myCAF phenotype and cancer cell stemness. In an in vivo murine xenotransplant model, Apt-1 inhibited MDA-MB-231 breast cancer growth and metastasis while also decreasing the expression of markers for the myCAF phenotype and cancer stemness. These results suggest that soluble Adam8 is a potential drug target and that Apt-1 is a novel agent directed against the extracellular Adam8 soluble metalloproteinase domain.

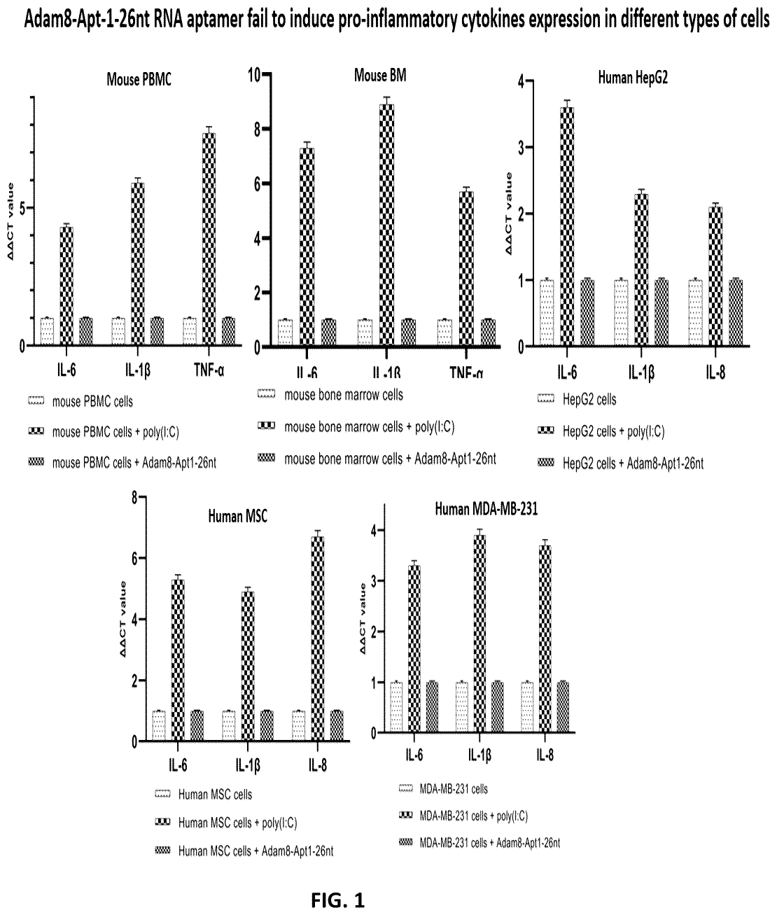

RNA aptamers represent a novel category of therapeutic agents^{8,9,17}. They are 12-80 nt ss RNA oligonucleotides with stable three-dimensional conformations that tightly and specifically bind to their target proteins. RNA aptamers bind to extracellular targets, such as the soluble Adam8 metalloproteinase domain. As demonstrated by Apt-1 and OPN-R3, these RNA aptamers typically exhibit binding affinities in the low nanomolar to picomolar range, are heat stable, are not immunogenic, and exhibit minimal batch-to-batch variability. (FIG. 1) Modifications, such as amino- or fluoro-substitutions at the 2′ position of pyrimidines, reduce the degradation of aptamers by nucleases. The biodistribution and clearance of aptamers are altered by the addition of polyethylene glycol and cholesterol. In addition, SELEX allows for selection from libraries consisting of up to 1015 ligands in order to generate high-affinity oligonucleotide ligands capable of binding to purified biochemical targets such as Adam8.

The potential underlying impediments to the clinical utility of aptamers include their susceptibility to nuclease degradation, renal filtration, and excretion; the potential for immunogenicity; and their assumption of altered in vivo structures that results in decreased function.

A recent search of clinical trials for the term “aptamer” yielded 53 current and past trials in which aptamers were tested as biosensor, imaging, or therapeutic agents for a variety of pathologies including bladder CA, COVID-19, HIV, age-related macular degeneration, CD30+ lymphoma and solid tumors, and metastatic colorectal and pancreatic cancers. Specifically in the realm of cancer therapeutics, two aptamers, namely, AS1411 and NOX-A12, have undergone clinical trials¹⁸. AS1411 is the first aptamer for the treatment of cancer in clinical application. In a phase I clinical trial, 17 patients with renal and non-small cell lung cancers with advanced solid tumors were treated with AS1411¹⁹. In the corresponding phase II trial, AS1411 was administered to 35 patients with metastatic renal cell carcinoma. The conclusion of these trials was that the anti-cancer effects of AS1411 were minor and that the toxicity profile was acceptable²⁰. NOX-A12 is a pegylated L-type RNA aptamer resistant to nuclease degradation that binds to chemokine CXCL12, which plays an important role in the TMEN and cancer cell signaling²¹. In the phase I/II clinical trials, 28 patients with CLL were treated with a combination therapy including NOX-A12. Consequently, 86% of patients had an overall response to treatment with a median progression-free survival of 15.4 months. No additional toxicity was associated with NOX-A12²².

Adam proteases are a group of membrane-bound enzymes with “sheddase” functions^{23,24}. Soluble ectodomain shedding of membrane proteins is an integral part of cell signaling in multiple settings, including cancer¹. Pro-tumorigenic effects have been associated with essential (Adam 10 and 17) and inducible proteases (Adam 8, 9, 12, 15, and 19). Adam8 was first identified in monocytic immune cells and subsequently demonstrated to be selectively expressed²³. Adam8 was initially thought to be immune-specific as the result of its induction via inflammatory signaling, including by tumor necrosis factor, lipopolysaccharide, interleukin-1, and interferon-γ. Studies in Adam8 KO mice indicate that Adam8 is not required for normal development and homeostasis²⁵.

Increased expression of Adam8 has been correlated with enhanced tumor growth and metastasis in breast, brain, pancreatic, liver, colon, and renal cancers¹. However, the role of Adam8 in cancer has not been well characterized. Adam8 is highly expressed in breast tumors, which is associated with an aggressive phenotype and poor patient outcomes^{26,27}. In primary breast tumors, Adam8-positive cells are most common in the invasion zone; Adam8 expression is maintained with metastases. Previous studies incorporating MDA-MB-231 Adam8 KO mouse xenograft models showed the presence of significantly smaller tumors, decreased levels of circulating tumor cells, and lower numbers of brain metastases²⁶. In hepatocellular carcinoma, high Adam8 expression is found in the majority of cases. Elevated Adam8 levels are associated with increased serum Alpha-fetoprotein (AFP), advanced tumor stage, poor differentiation, increased tumor recurrence and metastasis, and reduced survival^{28,29}. Adam8 KO in HepG2 cells exhibited reduced cell migration and invasion. Orthotopic murine xenograft models with Adam8 KO HepG2 presented significantly smaller tumors. Monoclonal antibody directed against Adam8 improved survival and reduced loss of body weight³⁰. Adam8 mAb also lowered AFP; slowed the progression of HCC; induced the expression of Casp3, Bax, and P53; and inhibited the expression of VEGF-A, PCNA, and Bcl2 in mouse livers. Adam8 has not been previously linked to the maintenance of the myCAF phenotype in the TMEs of breast or liver cancer.

Cancer growth and metastasis are regulated by reciprocal cross talk between the tumor microenvironment (TME) and cancer stem cells. The TME consists of highly complex and dynamic molecules, blood vessels, and various cells, which surround cancer cells. As one key TME component, myCAF carries out multiple functions in order to manipulate cancer development, such as facilitating extracellular matrix remodeling, accelerating angiogenesis, promoting cancer cells' epithelial-mesenchymal transition, increasing cancer cell invasion and metastasis, and facilitating the evasion of tumor immunosurveillance and therapeutic resistance. Although myCAF has been classified into subtypes based on cell surface markers or transcriptome profiling, there is no consensus regarding myCAF subtype classification and the subtypes' identification markers, so the most typical intracellular markers of tumor-promoting myCAF cells are α-SMA, Vim, and TNC. Cancer stem cells are functionally characterized by self-renewal and differentiation, which reprograms the TME to favor tumor initiation, heterogeneity, immune escape, invasion, metastasis, and therapeutic resistance. There is not a consensus marker for cancer stem cells (CSC); therefore, several pluripotent stem cell transcription factors, such as sox2, Oct4, and Nanog, are commonly applied to measure cancer stemness³¹.

Molecular drivers originating from the TME-CSC interaction network are ideal targets either in diagnostic or therapeutic clinical practice. The inventors' lab previously found that cancer cell-derived osteopontin (OPN), a matricellular protein, promotes bone-marrow-derived mesenchymal stem cells' (MSCs) resident transformation into myCAF, while maintenance is feedback-regulated by CSC stemness. Here, the inventors identified Adam8 as a sox2-dependent protein expressed in MDA-MB-231 breast cancer cells when cocultured with MSCs. The inventors previously found that myCAF-induced cancer stemness is required for the maintenance of the myCAF phenotype, suggesting that the initiation and maintenance of the myCAF phenotype required distinct cell-signaling crosstalk pathways between cancer cells and myCAF¹⁶. The inventors strategy was to isolate the cancer genes upregulated in the MSC coculture and downregulated in cancer (sox2-KD) when similarly cocultured with MSC. Adam8 was then identified as a candidate secreted protein induced by myCAF-mediated cancer stemness. Adam8 has a known sheddase function against which an RNA aptamer, Apt-1, was developed. The Apt-1-mediated blockade of the extracellular soluble Adam8 metalloproteinase domain abolishes the previously initiated myCAF phenotype (blocks the maintenance of the myCAF phenotype). In addition, cancer stemness is significantly decreased as a result, although previous studies have demonstrated that Apt-1 does not directly alter cancer stemness¹⁶. Xenograft models show that Apt-1 administration is associated with decreased tumor growth and metastasis, while flow cytometric analyses demonstrate significantly decreased fractions of myCAFs with Apt-1. The role of soluble Adam8 in the maintenance of the myCAF phenotype has not been previously characterized. The results described herein also suggest that the induction or initiation of the myCAF phenotype may be distinct from the maintenance of the myCAF phenotype.

The following non-limiting examples illustrate exemplary systems and components thereof in accordance with various embodiments of the disclosure. The examples are merely illustrative and are not intended to limit the disclosure in any way.

### EXAMPLE 1—CONSTRUCTION OF APT-1 AND METHOD OF TREATING CANCER

### Results

### Adam8, CA12, and CDH6 Selection

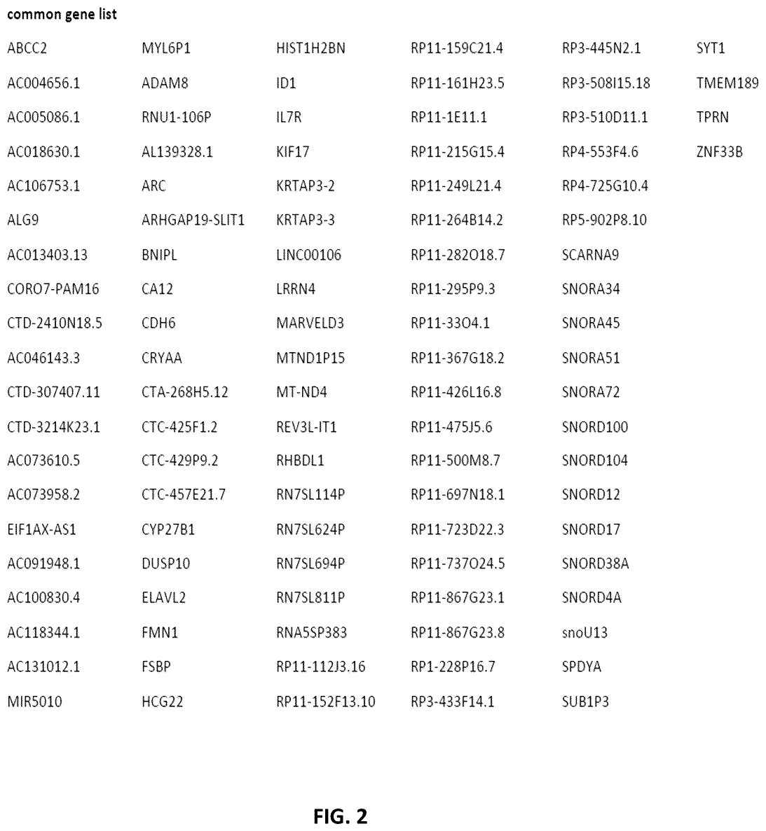

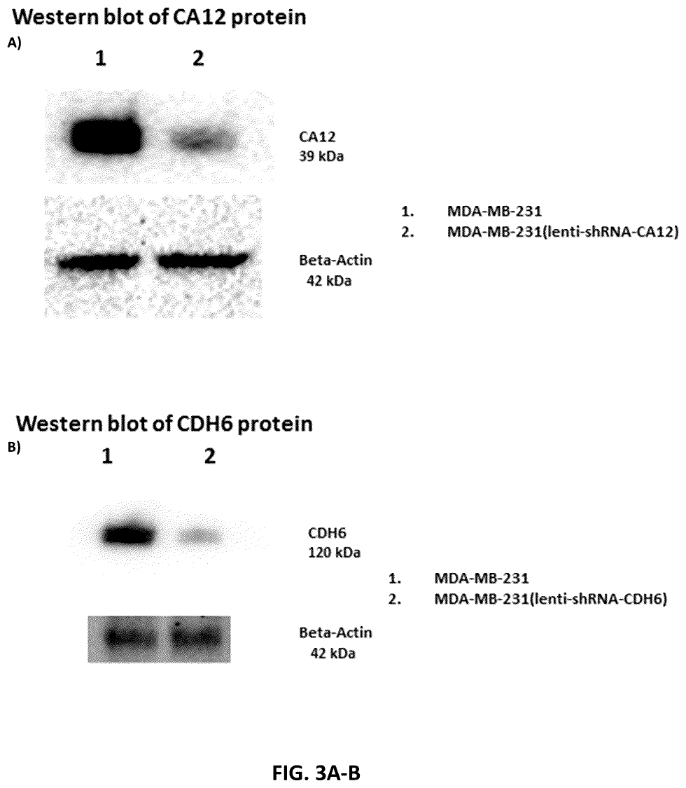

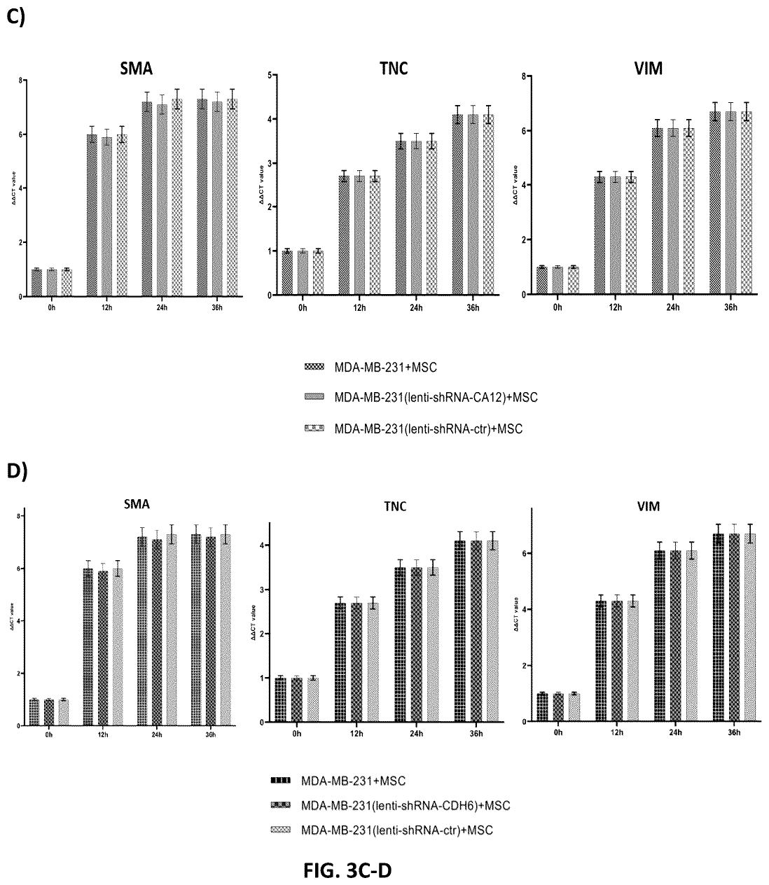

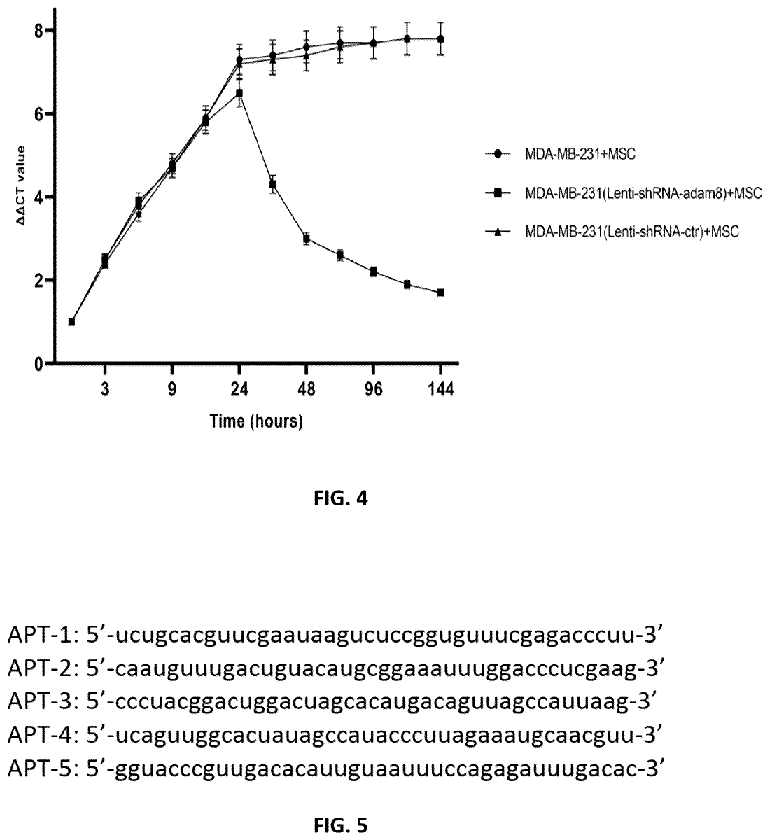

The inventors have previously found that myCAF-mediated cancer stemness is required for the maintenance of the myCAF phenotype in this system¹⁶. In the results of the current series of RNAseq analyses, Adam8, CA12, and CDH6 were identified as candidates for myCAF-induced cancer-stemness-related genes with secreted proteins. (FIG. 2) siRNA constructs targeting each candidate were transfected into MDA-MB-231 and then cocultured with MSCs. In comparison to parental MDA-MB-231+MSC cocultures, shRNA directed at CA12 and CDH6 did not significantly alter myCAF marker expression, α-smooth muscle actin (α-SMA), Vimentin (Vim), or Tenascin-C (TNC). (FIG. 3) Adam8-shRNA-transfected MDA-MB-231 cells did, however, result in the ablation of myCAF marker expression, indicating that Adam8 expression in cancer cells is required for the MSC development of the myCAF phenotype. (FIG. 4) The inventors then proceeded to develop an aptamer against Adam8.

### Synthesis and Characterization of Aptamer Targeting Adam8

Thirty clones were sequenced following the sixth round of SELEX, eleven of which were the Adam8 aptamer (Apt-1). (FIG. 5) The Adam8 RNA aptamer sequences tested herein were as follows:

Apt-1:
(SEQ ID NO: 1)
5′-UCUGCACGUUCGAAUAAGUCUCCGGUGUUUCGAGACCCUU-3′
Apt-2:
(SEQ ID NO: 2)
5′-CAAUGUUUGACUGUACAUGCGGAAAUUUGGACCCUCGAAG-3′
Apt-3:
(SEQ ID NO: 3)
5′-CCCUACGGACUGGACUAGCACAUGACAGUUAGCCAUUAAG-3′
Apt-4:
(SEQ ID NO: 4)
5′-UCAGUUGGCACUAUAGCCAUACCCUUAGAAAUGCAACGUU-3′
Apt-5:
(SEQ ID NO: 5)
5′-GGUACCCGUUGACACAUUGUAAUUUCCAGAGAUUUGACAC-3′

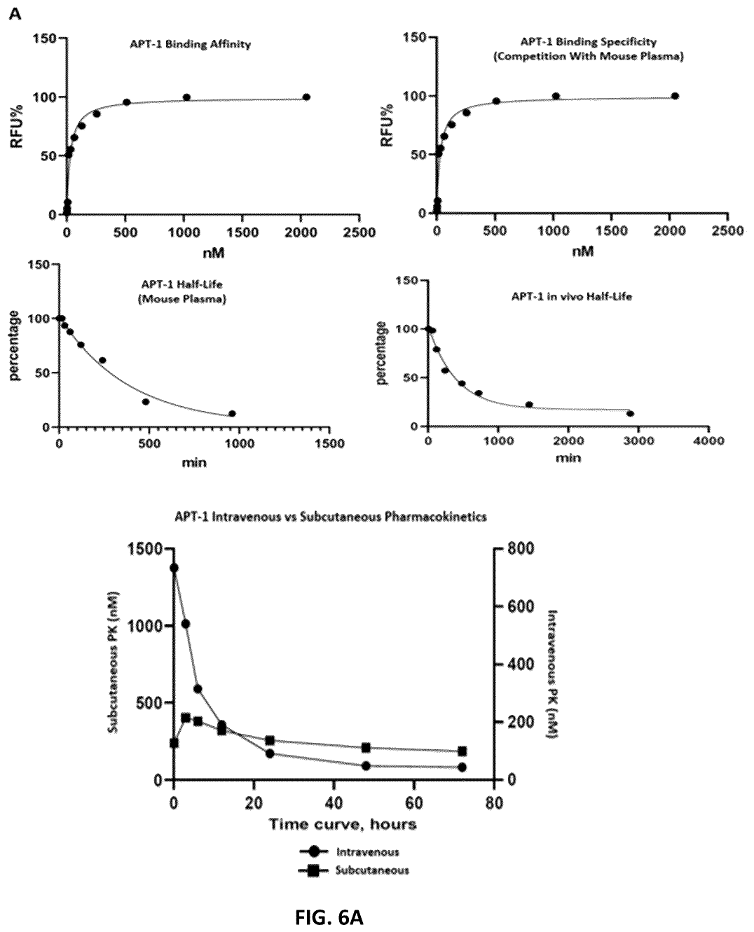

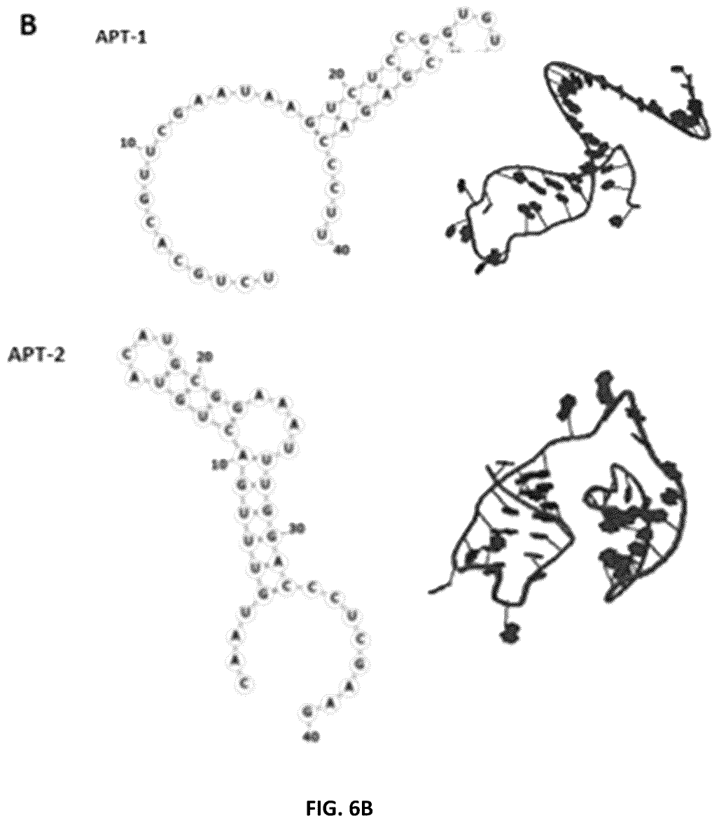

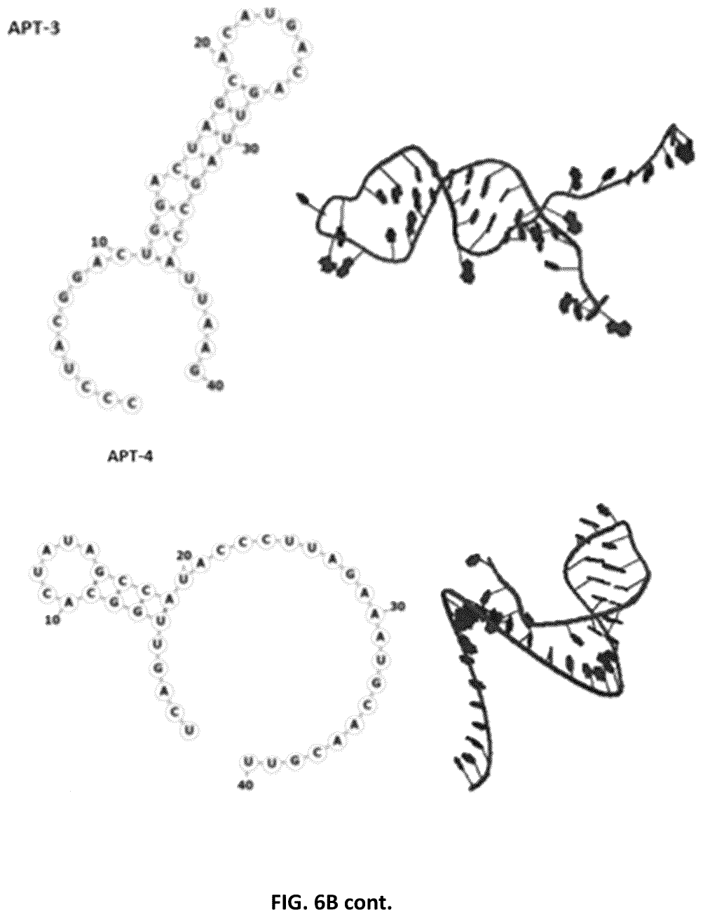

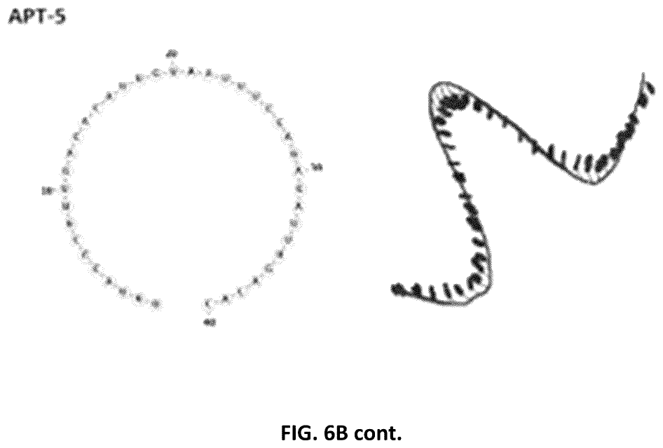

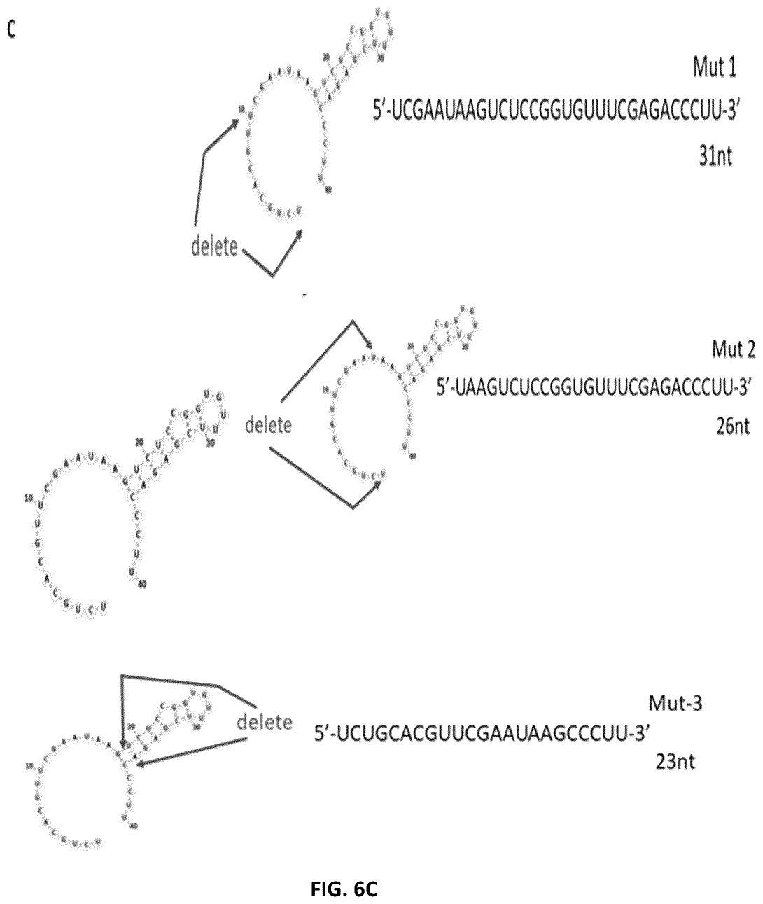

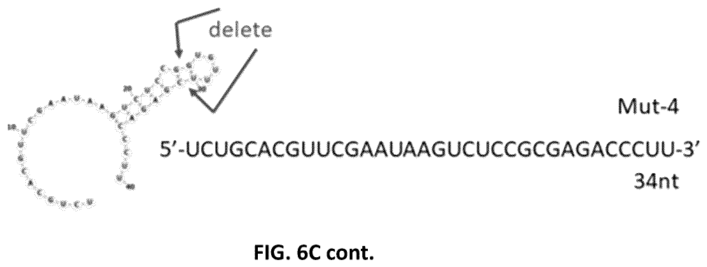

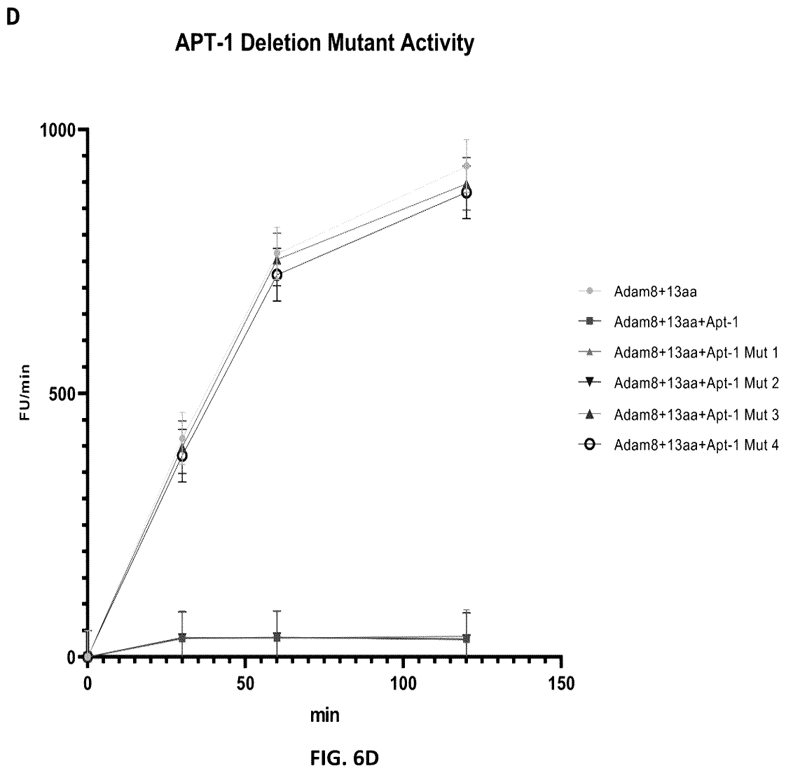

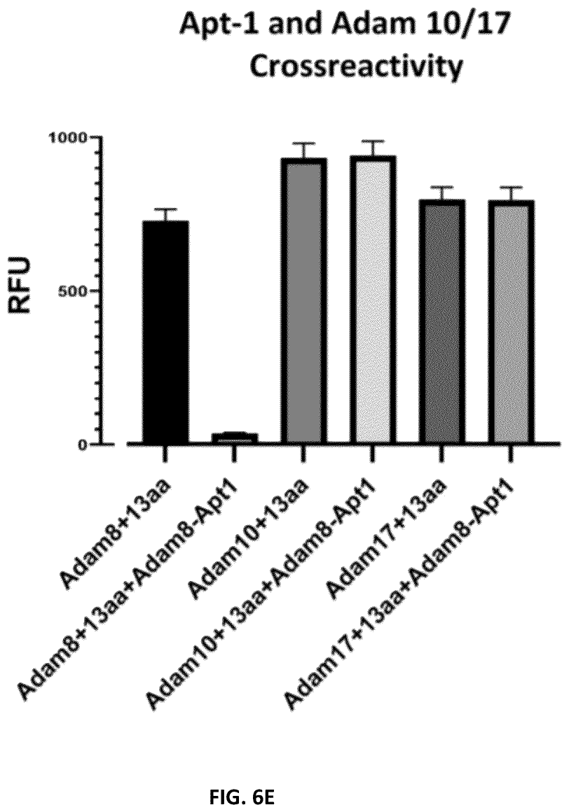

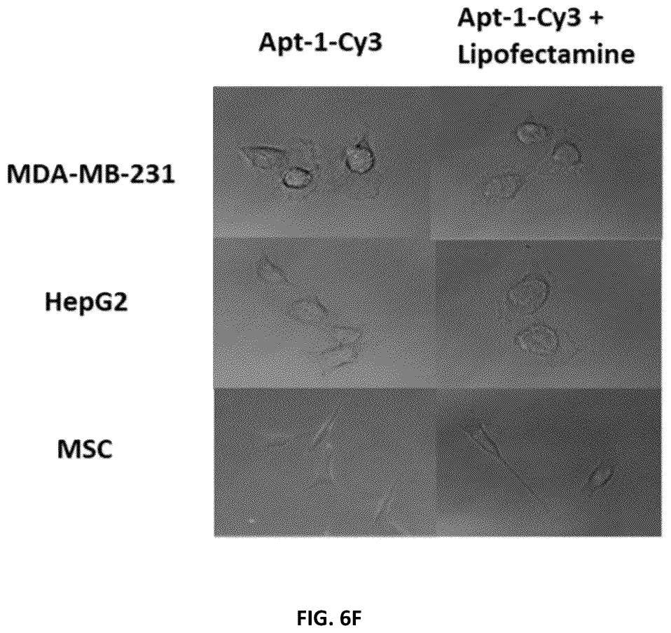

Pharmacodynamic and pharmacokinetic analyses were performed. The Kd values of Apt-1 and four other clones were determined; the Kd value of Apt-1 was the lowest at 29.7 nmol/L (19.25-44.12, 95% CI). (FIG. 6A) The in vitro half-life in mouse plasma was 267 min (174-471, 95% CI), while the in vivo half-life was 278 min (174-455, 95% CI). Pharmacokinetics for intravenous and subcutaneous delivery of Apt-1 demonstrated stable (82 nM and 99 nM, respectively) serum concentrations up to 72 h. The predicted secondary structure of Apt-1 contains the usual stem-loop structure of RNA aptamers and is shown in FIG. 6B along with those of APT-2, 3, 4, and 5. The mutagenesis of Apt-1 was then induced to determine the active site. (FIG. 6C) Deletion constructs were then tested using in vitro recombinant human Adam8 soluble domain activation and metalloproteinase activity assays. Deletion constructs had the following sequences:

Mut 1 (31 nt):
(SEQ ID NO: 6)
5′-UCGAAUAAGUCUCCGGUGUUUCGAGACCCUU-3′
Mut 2 (26 nt) (aka Apt1-1-26nt):
(SEQ ID NO: 7)
5′-UAAGUCUCCGGUGUUUCGAGACCCUU-3′
Mut 3 (23 nt):
(SEQ ID NO: 8)
5′-UCUGCACGUUCGAAUAAGCCCUU-3′
Mut 4 (34 nt):
(SEQ ID NO: 9)
5′-UCUGCACGUUCGAAUAAGUCGCCGCGAGACCCUU-3′

The results demonstrate that only Apt-1 remained active. (FIG. 6D) Adam10 and Adam17 cross-reactivity with Apt-1 was determined. (FIG. 6E) The coincubation of Apt-1 with Adam10 or Adam17 in the presence of the 13aa substrate did not impact Adam10 and Adam17 metalloproteinase activity. Apt-1 was linked to Cy3 and incubated with MDA-MB231, HepG2, and MSC in the presence and absence of lipofectamine to selectively block Adam8 metalloproteinase activity. (FIG. 6F)

### In Vitro Activity of Apt-1

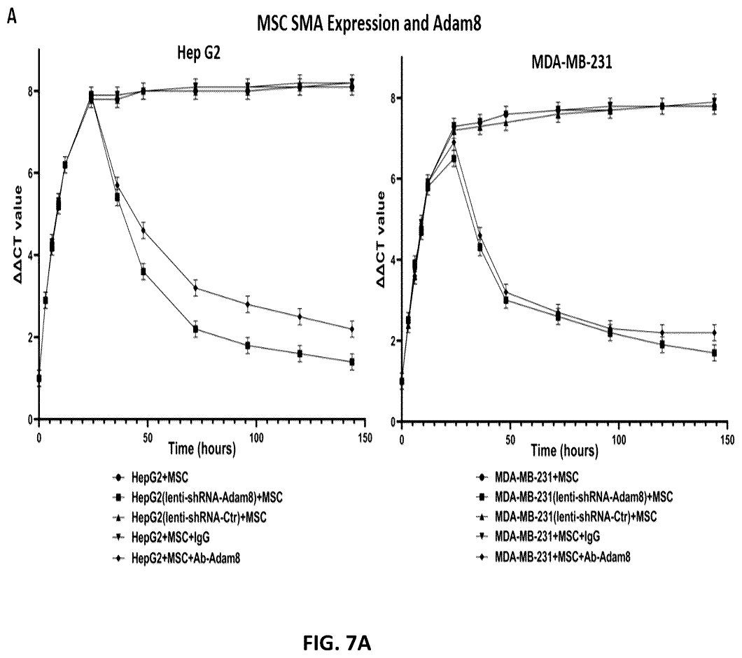

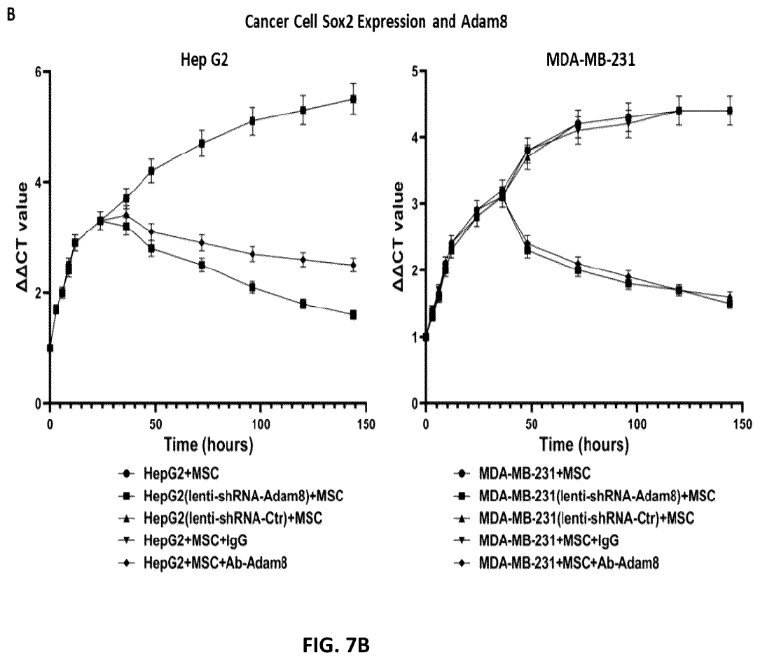

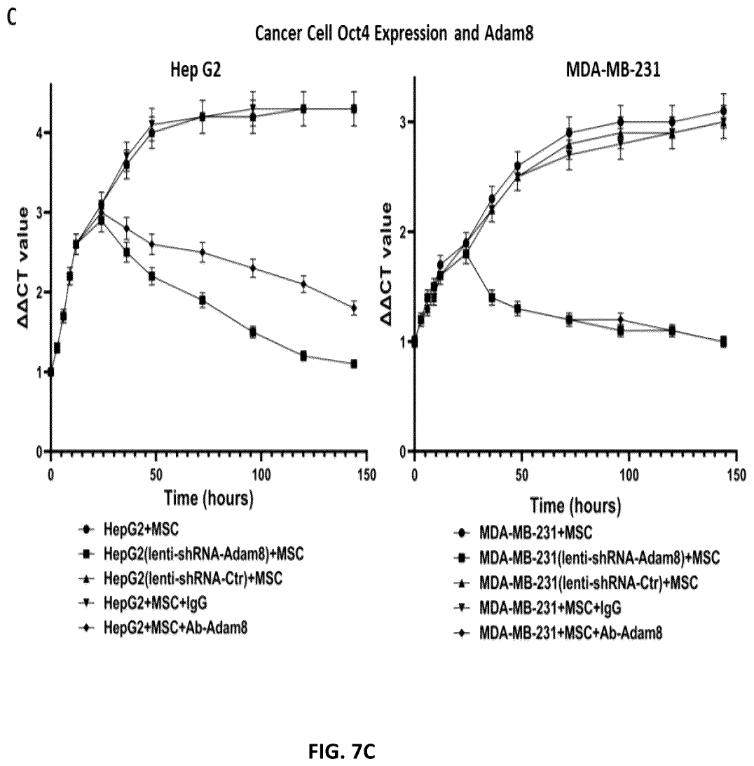

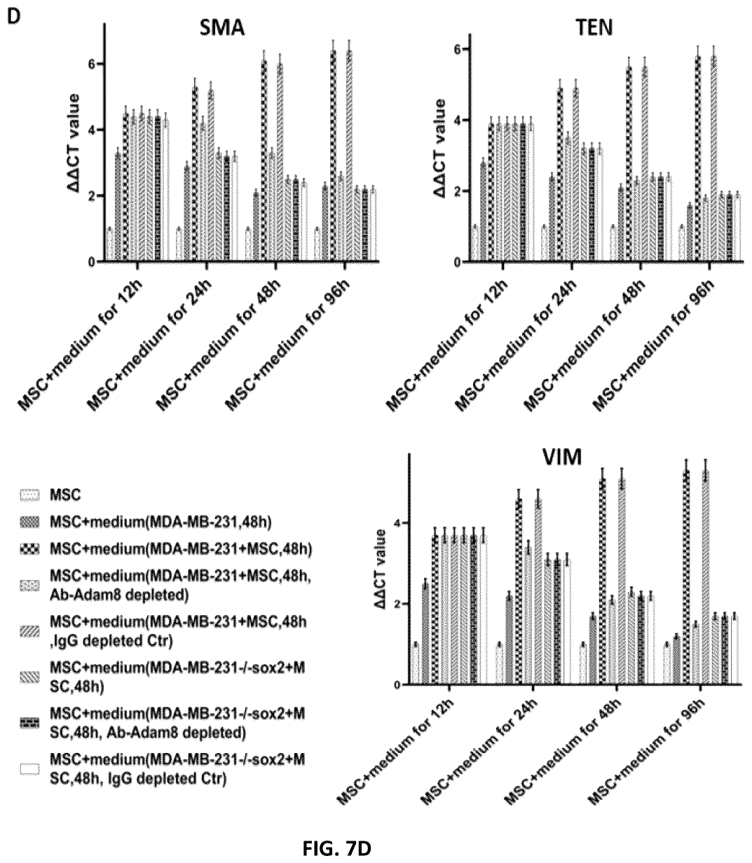

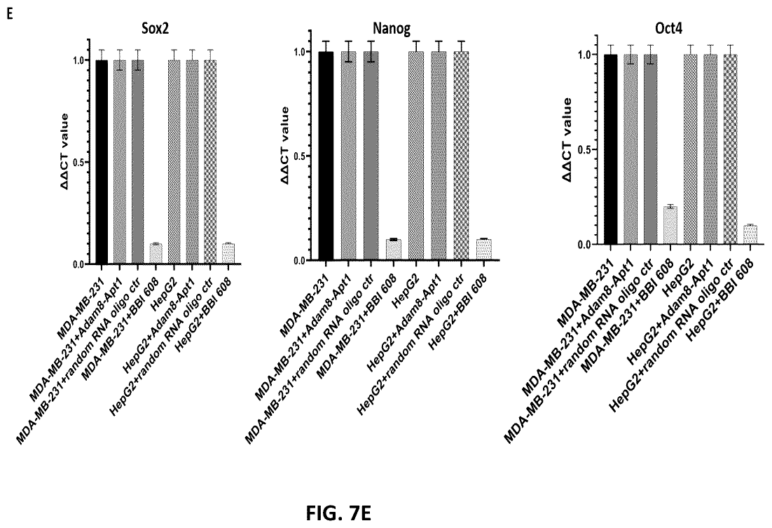

To determine the potential role of Adam8 in the cell-signaling crosstalk between MSCs and cancer cells toward the induction of the myCAF phenotype with a concomitant increase in cancer stemness, the inventors performed coculture studies in which human MSC cells were cultured with either MDA-MB-231 breast cancer cells or HepG2 liver cancer cells and the Adam8 antibody added at different time points to block Adam8 bioactivity. In selected instances, MSCs were cultured with MDA-MB-231 (Adam8-KD) or HepG2 (Adam8-KD), and in others, Adam8 mAb was added. (FIG. 7A) After coculturing for periods of up to 144 h, MSC α-SMA expression was measured via real-time PCR as a reflection of the myofibroblast myCAF phenotype (MDA-MB-231 alone or HepG2 alone were used as reference samples). In all instances, the coincubation of cancer cells with MSCs resulted in increased α-SMA expression with a plateau at 30 h, except in the presence of Adam8 mAb or lentivirus Adam8 KO, in which there was a progressive decline in α-SMA expression starting at approx. 30 h and ranging to a level 1.5-2-fold greater than the baseline. The inventors also determined the HepG2 and MDA-MB-231 expression of the cancer stemness markers sox2 and Oct4 in the coculture models. (FIGS. 7B and C) In a manner similar to that presented for α-SMA, the cancer cell expression of both stemness markers increased with a plateau at 30 h, except in instances wherein Adam8 mAb or lentivirus Adam8 KO were present. These results indicate that Adam8 is required for the maintenance of the myCAF and cancer stemness phenotype in these coculture models.

Conditioned media studies were then performed. MSCs were cocultured alone, with MDA-MB-231, or with MDA-MB-231 (sox2-KD) for 48 h. In selected instances, the medium was supplemented with Adam8 mAb to generate Adam8-depleted media or an IgG control. The media were then transferred to MSCs, and myCAF markers were determined after 12, 24, 48, and 96 h. (FIG. 7D) In the MSCs exposed to the MDA-MB231 medium, the levels of myCAF markers peaked at 12 h, with a progressive decline thereafter. In contrast, MSCs exposed to the MSC+MDA-MB-231-primed medium expressed myCAF markers in a progressively increasing manner with a near plateau at 96 h. This pattern was ablated in the MSCs exposed to the MSC+MDA-MB-231 medium for which Adam8 had been depleted, presenting a myCAF expression pattern similar to that of the MDA-MB-231 media. In MSC exposed to media from MSC+MDA-MB-231 (sox2-KD), myCAF markers peaked at 12 h with progressive decline afterwards regardless of the presence or absence of Adam8. As controls, MDA-MB-231 or HepG2 were cultured with Apt-1 and/or the pharmacologic stemness inhibitor BBI 608. (FIG. 7E) In this setting, Apt-1 does not directly alter cancer stemness marker expression. These results suggest that Adam8 in the extracellular space (and cancer stemness) are required for the maintenance of the myCAF phenotype following initial induction.

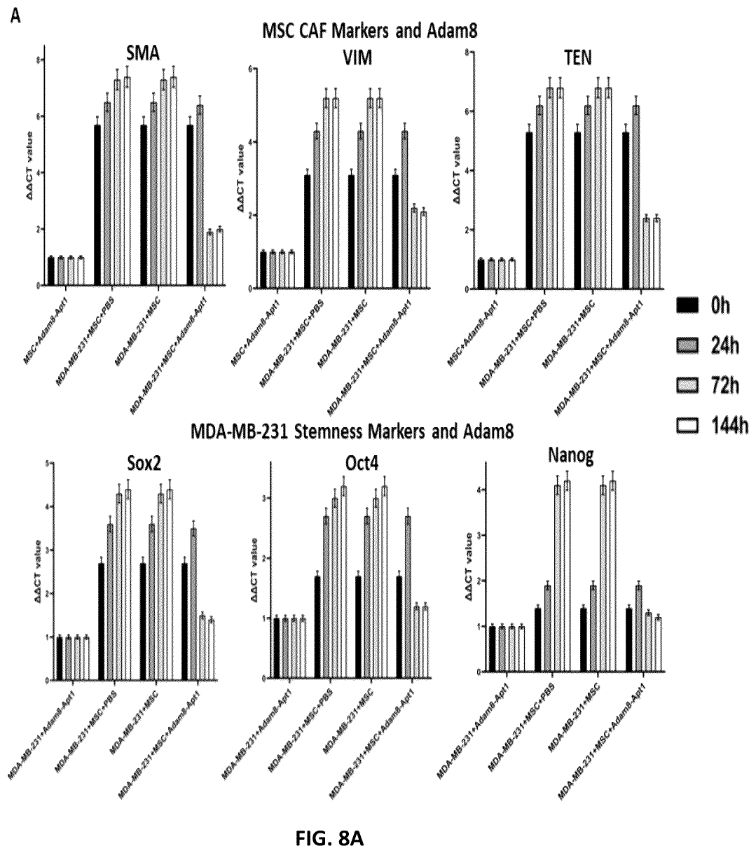

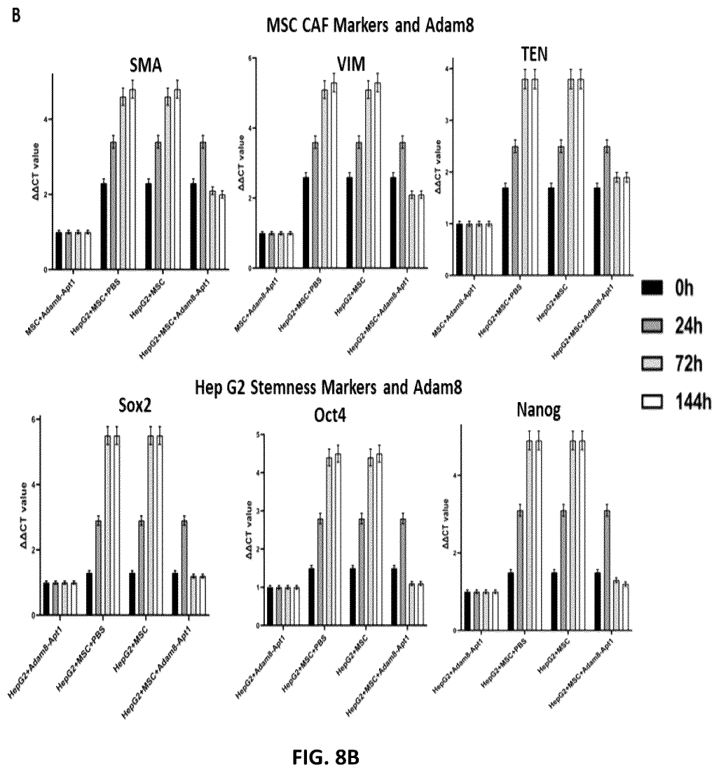

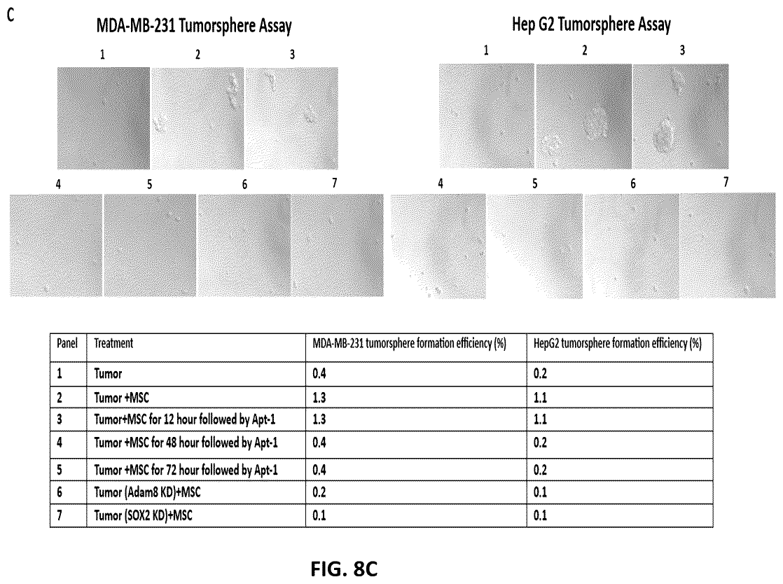

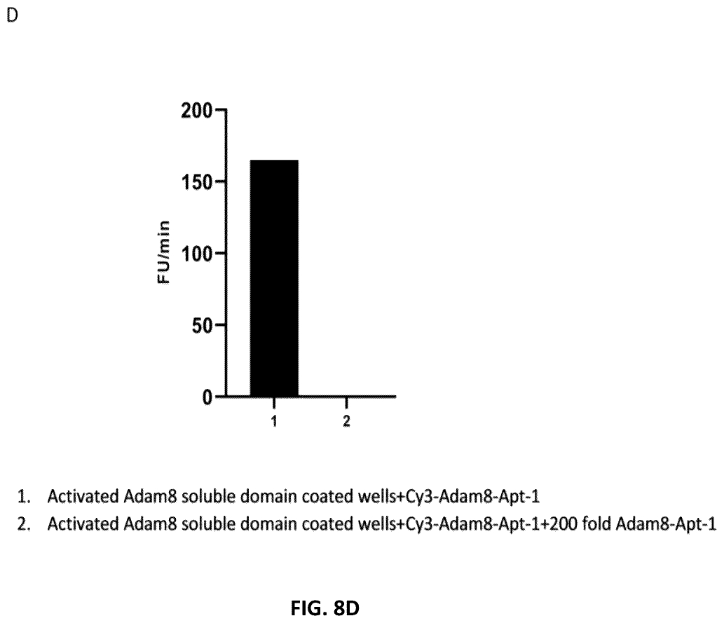

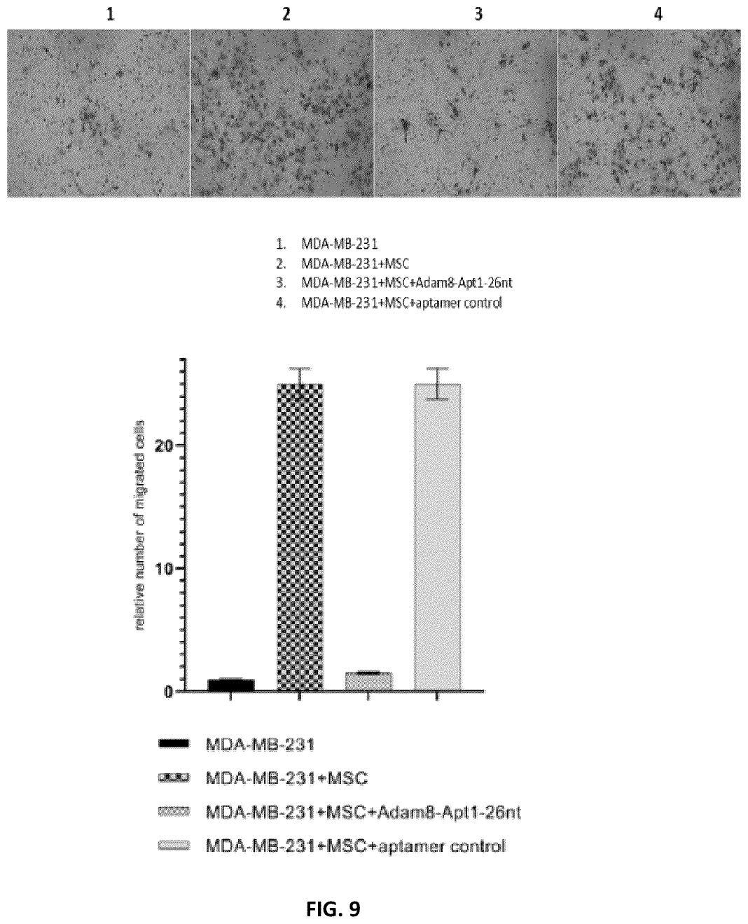

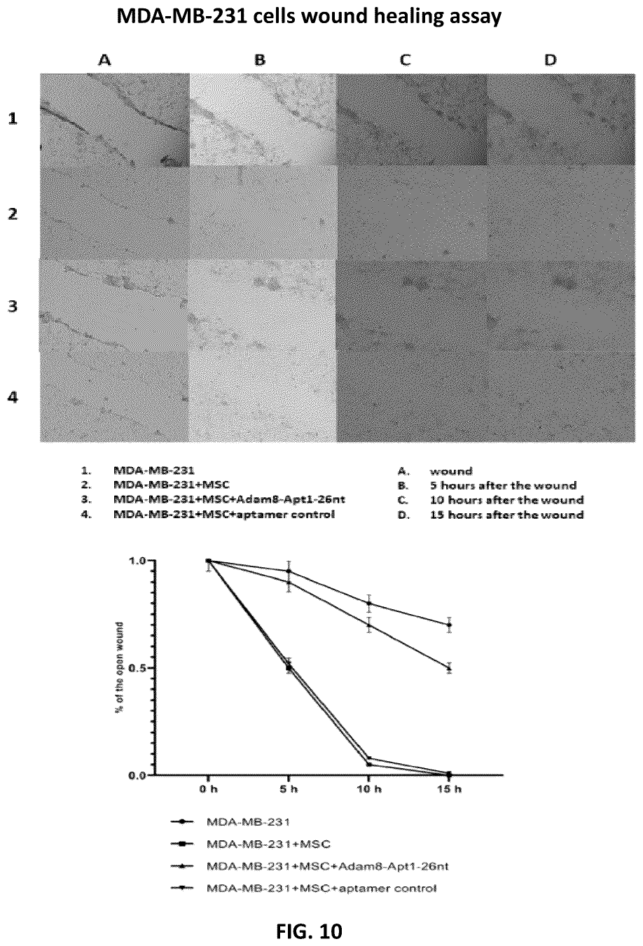

Apt-1 activity was then assessed using the same coculture system. (FIGS. 8A and B) In these studies, the levels of myCAF phenotype markers, α-SMA, TenC and Vim, and the cancer stemness markers sox2, Oct4, and Nanog were measured in the MSCs and cancer cells. While the levels of both myCAF and stemness markers were increased following coculture, the administration of Apt-1 was associated with lowered expression of both after a peak at 24 h in both MDA-MB-231 and HepG2 cells. To functionally confirm Apt-1 inhibition of cancer cell stemness, the inventors performed a tumorsphere formation assay with MDA-MB-231 or HepG2 cells co-cultured with MSCs. (FIG. 8C) Apt-1 treatment at 48 h and 72 h co-culture time points significantly decreased tumorsphere formation efficiency. These results indicate that Apt-1 blocked the maintenance of the myCAF and cancer stemness phenotypes in the coculture models. (FIGS. 9 and 10)

Apt-1 pulldown studies were performed based on binding competition. The His-tag-labeled activated human Adam8 soluble domain was coated on Ni-NTA-96-well plates and incubated with Cy3-labeled Apt-1 only or with a His-tag-labeled and-non-labeled Apt-1 mixture (1:200) to determine the degree of binding competition. The results showed that Cy3 intensity was completely abolished in the Apt-1 mixture binding group. (FIG. 8D) The inventors also performed in vitro Adam8 soluble domain metalloproteinase activity assays. After applying an internally quenched, MCA/Dpa-labeled, Adam8 fluorogenic substrate (13aa) with or without the addition of Apt-1 or Apt-1 deletion mutants, the results indicate that Apt-1 can effectively inhibit human Adam8 soluble domain metalloproteinase in vitro. Overall, these studies confirmed Apt-1's binding to the shed extracellular soluble Adam8 and the inactivation of its metalloproteinase activity.

### In Vivo Activity of Apt-1 Against Established Tumor

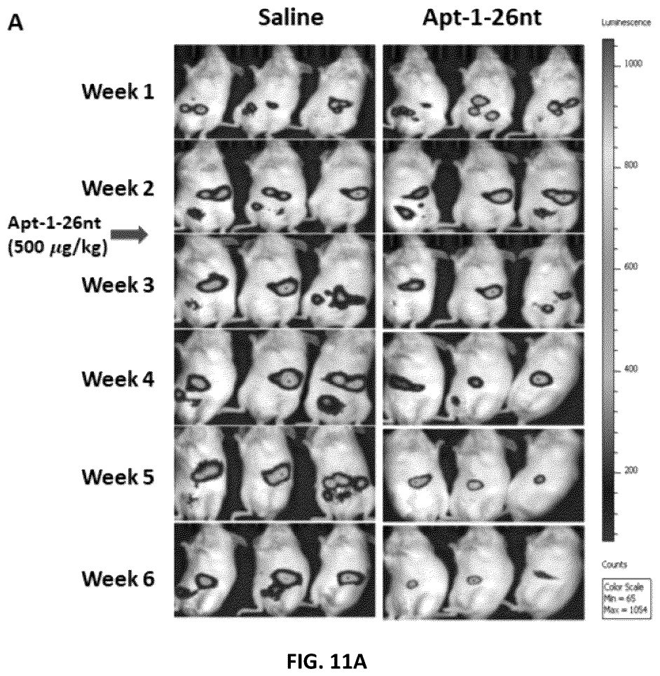

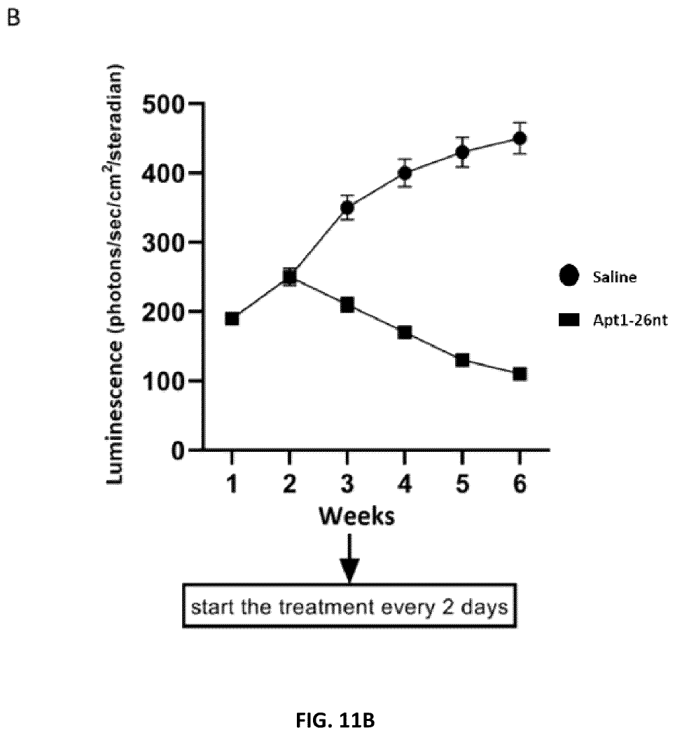

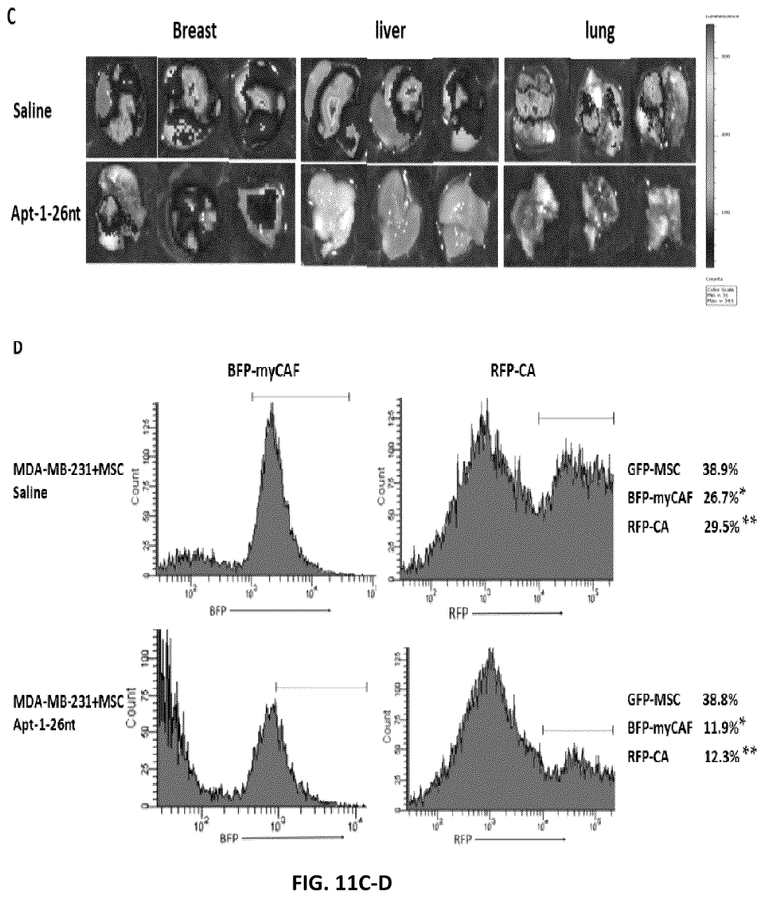

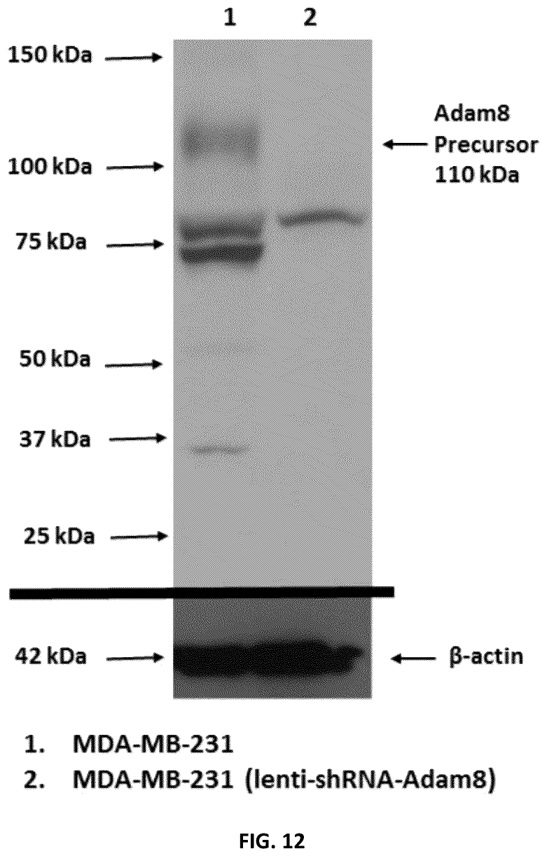

The inventors then tested the efficacy of Apt-1-26nt (also referred to as “Mut 2” herein) in an in vivo NOD-scid mouse model of human breast cancer. (FIG. 11) The R4 mammary fat pads of 6-week-old female NOD SCID mice were injected with 10⁶ MDA-MB-231 cells expressing luciferase/RFP along with MSCs expressing GFP and α-SMA promoter-inducible BFP. Three mice were used per group. Three weeks after inoculation, treatment was initiated with Adam8 Apt-1-26nt (500 μg/kg or saline control via tail vein injection every 2 days). The images demonstrate that Apt-1-26nt administration is associated with the stabilization of tumors with minimal growth. (FIG. 11A) In contrast, the cocultures of MDA-MB-231 cells (Adam8-KD) with MSC were not sustained beyond the first week. Luciferase activity corroborated the observation that Apt-1-26nt stabilized and/or decreased tumor growth. (FIG. 11B) Following sacrifice at 6 weeks, the primary tumors, livers, and lungs were examined with respect to luciferase activity. These images indicate that there were no tumor metastases in the livers and lungs of the Apt-1-26nt-treated animals. (FIG. 11C) Flow cytometry was performed on the explanted tumors. (FIG. 11D) The Apt-1-26nt-treated animals exhibited significantly fewer myCAF and cancer cells compared to the saline-treated controls. These data suggest that Apt-1-26nt inhibits the growth of established tumor cells in vivo in parallel with decreased amounts of myCAF.

### Materials and Methods

### Human-α-SMA-Promoter-Driven BFP Reporter in Human MSC Cells

The pCDH-CMV-EF1-Puro lentiviral vector containing human α-SMA promoter (262 bp) with an enhancer (123 bp) was kindly provided by Dr. Shading Bao's lab (Lerner Research Institute, Cleveland, OH, USA). The mcherry coding region was replaced with the BFP coding region cloned from the plasmid of pCAGGs-BFP (plasmid#127348, Addgene, Watertown, MA, USA) and confirmed by DNA sequencing. The Lentivirus Transduction Enhancer kit (GenTarget Inc., San Diego, CA, USA) was used to generate human-α-SMA-promoter-driven BFP reporter in human MSC cells¹⁰.

### Cell Culture

Human mesenchymal stem cells (MSCs) were obtained from the Texas A&M Institute and maintained in Minimal Essential Medium (MEM) media with 20% fetal bovine serum. Human breast cancer cells MDA-MB-231 were obtained from ATCC (Manassas, VA, USA) and maintained in Leibovitz's L-15 medium (ATCC 30-2008). All cells were cultured in 5% CO₂ incubator at 37° C. MDA-MB-231 cells were transfected with Sox2 shRNA lentiviral particles (Santa Cruz Biotechnology, Dallas, TX, USA, sc-38408-v) to constitutively knockdown Sox-2 for use in the co-culture system (confirmed via both real-time PCR and Western blot).

### Co-Culture

For all co-culture experiments, tumor cells and MSCs in a 1:1 ratio were plated in Boyden Chamber (Corning Inc., Corning, NY, USA) wells with 0.4 μm pores that allow cytokine and growth factor passage but prevent cell movement.

### Whole-Transcriptome Sequencing

The total RNA from MDA-MB-231 cells or MDA-MB-231 (Sox2-KD) cells in co-culture was extracted with RNeasy mini kit (QIAGEN, Germantown, MD, USA) according to the manufacturer's protocol. The total RNA was sent to NUSeq Core facility of Northwestern University to perform RNA sample integrity assessment, cDNA library preparation, whole-transcriptome sequencing (50 bp; paired-end; 300 M Read, Evanston, IL, USA, and data analysis. Triplicate total RNA samples were extracted from MDA-MB-231 or MDA-MB-231 (Sox2-KD) cells of the following groups:
1. MDA-MB-231 (72 h culture);
2. MDA-MB-231+MSC (72 h co-culture in the Boyden Chamber);
3. MDA-MB-231 (Sox2-KD)+MSC (72 h co-culture in the Boyden Chamber).
   Profiling of myCAF-Induced Cancer-Stemness-Related Genes

The inventors first identified myCAF-dependent gene expression in cancer cells by comparing group 2 to group 1 in terms of increased or newly expressed genes (p<0.05). The myCAF-induced sox2-dependent genes was identified by determining which genes decreased or were no longer present by comparing group 3 to group 2 (p<0.05). There are 104 genes common to the two comparison groups, thus rendering them myCAF-induced c ancer-stemness-related genes. Profiling of secreted myCAF-induced cancer-stemness-related genes: By searching these 104 genes on the human cancer secretome database (176.58.113.186), the inventors identified 9 genes that encode secreted myCAF-induced cancer-stemness-related proteins. By searching for these 9 genes in a sox2-regulated gene expression database, the inventors narrowed the search to 3 genes (Adam8, CA12 and CDH6) representing strong candidates for secreted myCAF-induced cancer-stemness-related genes.

### Aptamer Selection

The inventors have previously published detailed protocols in which a DuraScribe kit (Biosearch Technologies, Petaluma, CA, USA) and a 40 bp DNA aptamer library (Alpha Diagnostic International, San Antonio, TX, USA) were used to generate an RNA pool¹¹. Recombinant human Adam8 protein (Ile-17 to Pro497, Acro Biosystems, Newark, DE, USA) was processed with thermolysin cleavage in vitro to remove its Pro-domain in accordance with the manufacturer's manual. The his-tag c-terminal-labeled human Adam8 metalloproteinase domain was applied in the aptamer SELEX selection. A negative selection to remove filter-binding aptamers was performed through Nitrocellulose filter (0.45 μm, Schleicher & Schuell, Keene, NH, USA) incubated with the RNA pool in PBS buffer at 37° C. for 4 h. Under the same conditions, 5 μM protein and 50 μM RNA pools were incubated for 4 h, and the protein/aptamer complex was recovered through the filter flow and using phenol/chloroform extraction/ethanol precipitation. For each round of selection, the protein/aptamer binding affinity was quantified using a competition assay.

### Binding Affinity Assays

Ni-NTA-coated 96-well plates were coated with activated recombinant human Adam8 soluble domain his-tag protein. Cy3-labeled Adam8 aptamers were synthesized via IDT (Integrated DNA Technologies, Coralville, IA, USA). Cy3-Adam8 aptamers were added to the Adam8-Ni-NTA plates at different concentrations in PBS solution at room temperature for 30 min, washed 3 times with PBS, and quantified with Cytation 1 (BioTek, Santa Clara, CA, USA)¹²

Adam8, CA12, and CDH6 genes' Knockdown in MDA-MB-231 Cells

Human breast cancer MDA-MB-231 cells were transfected with Adam8/CA12/CDH6 siRNA mixture or their individual shRNA lentiviral particles (Santa Cruz Biotechnology, sc-41406/v, sc-41463/v, sc-29383/v) and co-cultured with MSC cells as described above. α-SMA gene expression was quantified with RT-PCR.

MSC Treated with Adam8 Immunodepletion Medium

The human Adam8 antibody (R&D Systems, Minneapolis, MN, USA, AF1031) was used to immunodeplete the Adam8 soluble domain from the co-culture medium of MDA-MB-231 and MSC (48 h) and was then applied to MSC cells for culturing at different time points. Adam8 antibody (1:100 dilution) or goat IgG control were added to the collected co-culture medium for overnight incubation at room temperature, and protein A-agarose was added and incubated for 2 h on a roller system at 4° C. The supernatants were collected and used to treat MSC. The total RNA was harvested from MSC cells at different time points, and gene expression of α-SMA/Ten-C/Vim was quantified using RT-PCR as described previously¹³.

### In Vitro Recombinant Human Adam8 Soluble Domain Activation and Metalloproteinase Activity Assay

Recombinant human Adam8 soluble domain (Met1-Pro497) protein and its fluorogenic substrate (13aa) were ordered from R&D systems (Minneapolis, MN, USA). The Adam8 soluble domain activation and metalloproteinase assay was performed as per the manufacturer's protocol. Briefly, recombinant human Adam8 soluble domain (Met1-Pro497) protein was diluted to 400 μg/mL in TCN assay buffer (50 mM Tris, 10 mM CaCL2, 150 mM NaCL, and pH 7.5). After adding an equal volume of 1.5 μg/mL thermolysin and incubation at 37° C. for 30 min, the reaction was stopped by adding Phosphoramidon to a final concentration of 0.05 mM at room temperature for 15 min. The activated recombinant human Adam8 soluble domain protein was diluted to 40 ng/μL in TCN assay buffer in the presence or absence of Adam8 aptamers at different concentrations and mixed for 1 min. The fluorogenic substrate was added at 37° C. and quantified with Cytation 1 (excitation: 320 nm; emission: 405 nm).

### In Vitro Binding Confirmation of Adam8 Extracellular Domain and Apt1 Via Binding Competition Assay

This protocol has been described previously by Mahajan¹², and is incorporated herein in its entirety. Briefly, Ni-NTA-coated 96-well plates were coated with activated recombinant human Adam8 soluble domain his-tag protein (Ile17-Pro497, Acro Biosystems, Newark, DE, USA). Cy3-labeled Adam8-Apt1-26nt was synthesized via IDT (Integrated DNA Technologies, Coralville, IA, USA). A total of 10 pmol of Cy3-labeled Adam8-Apt1-26nt with or without 2 nmol of unlabeled Adam8-Apt1-26nt in 50 μL PBS was added to the above wells. After binding for 1 h and 3 washes with PBS, Cy3 intensity in each well was quantified using Cytation 1 (BioTek, Santa Clara, CA, USA).

In Vivo Pharmacokinetics (PK), Intravenous Vs Subcutaneous (IV Vs. SC) Injection, and in Vivo Half-Life Quantification

6-week-old female NOD SCID mice were used. The dosing schedules are listed below in Table 1. Adam8 aptamer concentration was quantified using RT-PCR.

TABLE 1
Dosing schedules.

| | PK | IV vs. SC | In Vitro Half-Life |
|---|---|---|---|
| injection location | tail vein | neck | 0.5 pg + 50 μL mouse |
| aptamer amount | 2.2 nM | 2.2 nM | plasma incubation at |
| | | | 37° C. in 5% CO₂ |
| blood harvest | 3, 6, 12, | 3, 6, 12, 24, | 3, 6, 12, 24, 48 h |
| time points | 24, 48 h | 48, 72 h | |

### Evaluation of Aptamer Intracellular Uptake

4×10⁵ MDA-MB-231 cells, HepG2 cells, or MSC cells were seeded on 12-well plates to perform transfection with 80 μmol/L Cy3-labeled Adam8 aptamers and OptiMEM medium or with Lipofectamine 2000 packed aptamer and OptiMEM-positive controls. Images were taken at 72 h using a Leica SP2 confocal microscope.

### Tumorsphere Formation Assay

A detailed protocol conducted in accordance with the Millipore Sigma manual was used, which has been described previously¹⁴, and is incorporated herein in its entirety. Briefly, the cancer cells were trypsinized into the single-cell suspension with Trypsin-EDTA (Sigma, St. Louis, MO, USA, T3924) for 2-4 min at room temperature, pipetted up and down 20 times using 1 ml tip, and two volumes of trypsin inhibitor solution (Sigma, T6414) were added to stop trypsin activity. The single-cell suspension was plated at 200 cells per cm2 with 3D tumorsphere Medium XF (Sigma, C-280700) on Corning Costar ultra-low attachment 6-well plate (Sigma, CLS3471). Equal amounts of MSC were added to the 6 well-plate insert (0.4 μm) and placed on the top. Adam8-Apt-1-26nt (3 μM) was added to the medium at 12 h, 48 h, and 72 h co-culture time points, respectively (daily). On day 7, images were acquired using Zeiss Apo Tome 2 microscope (10×20).

### Xenograft Regression Model

All animal-handling activities and other procedures were approved by the University of South Florida Animal Care and Use Committee. A total of 1×10⁶ tumor cells w/wo MSC cells mixed with 50% Matrigel were implanted into 6-week-old female NOD SCID mice (Charles River, Wilmington, MA, USA) at the R4 location on their mammary fat pads. Bioluminescence imaging was performed weekly using an IVIS 100 imaging system (Xenogen, Hopkinton, MA, USA). Total photon counts (1 min) or counts/second were obtained. Two weeks after the cell implantation procedure, the mice were treated through tail vein injection with aptamer or saline control every two days until eight weeks had passed.

### Fluorescence-Activated Cell Sorting

Fresh primary tumors were obtained. Single-cell suspensions were prepared as reported previously². The tissues were finely minced with surgical scissors and transferred to 10 mL collagenase-PBS solution (1×PBS, PH7.4; 0.025% collagenase, 0.05% pronase, and 0.04% DNase I). After 1 h incubation at 37° C., the tissue pellets were centrifuged at 300 g for 10 min at 4° C. and washed three times with 5 mL of PBS. The tissue homogenate was gently passed through a 70 μm pore nylon mesh filter at 4° C. Cells were sorted using BD FACSMELODY (BD Biosciences, Franklin Lakes, NJ, USA). For GFP-positive cells sorting, cells were excited using a 488 nm laser, with emission data collected through a 530/30 band-pass filter. For RFP-positive cell sorting, cells were excited using a 561 nm laser, with emission data collected through a 610/20 band-pass filter. For BFP-positive cell sorting, cells were excited using a 405 nm laser, with emission data collected through a 440/50 band-pass filter. The sorted cells were collected in PBS and stored at −80° C.¹⁵.

For RT-qPCR, GAPDH was used as the endogenous housekeeping gene, and delta-delta Ct analysis was performed. Pharmacokinetic and pharmacodynamic calculations were performed using GraphPad Prism version 9.1.1 (226) software (San Diego, CA, USA).

### Mouse Peripheral Blood Mononuclear Cell (PBMC) Isolation and Adam8-Apt1-26nt Treatment

The Ficoll-Paque density gradient centrifugation-based method used to isolate PBMC was described previously¹. Briefly, 1 ml of anticoagulant-treated mouse blood was mixed with the same volume of RPMI 1640 media. This diluted blood sample was loaded onto 3 ml of Ficoll-Paque media (1.076 g/ml); then, it was centrifuged at 400 g ×30 min at 20° C., the upper layer was carefully discarded, and the lower layer was transferred to a new tube. The cells were washed with three volumes of RPMI 1640 media and centrifuged at 400 g×10 min at 20° C. The cell pellet was resuspended with RPMI 1640 containing 10% Fetal Bovine Serum and treated with 3 uM Adam8-Apt1-26nt for 24 h in 5% CO₂ incubator at 37° C. Both adherent and suspension cells were harvested, and total RNA isolation was performed.

### Mouse Bone Marrow Cells' Isolation

Mouse femur and tibia were dissected and flushed with RPMI 1640 media containing 10% fetal bovine serum. The flushed bone marrow cells were treated with 3 uM Adam8-Apt1-26nt for 24 h in a 5% CO₂ incubator at 37° C. Both adherent and suspension cells were harvested, and total RNA isolation was performed.

### EXAMPLE 2—METHOD OF TREATING BREAST CANCER (PROPHETIC)

A 49 year old female patient presents with a new lump in her right breast. The lump is biopsied and the patient is diagnosed with breast cancer. The patient is administered a therapeutically effective amount of a composition comprising an RNA aptamer having the sequence of SEQ ID NO:7. The mouse studies are used to determine a therapeutically effective administration dose, frequency, and treatment length. The patient's tumor is monitored during the treatment regimen. At the conclusion of the treatment regimen, the patient is evaluated and it is noted that the tumor has not grown and has decreased in size and there is no indication of tumor metastasis.

### EXAMPLE 3—METHOD OF TREATING LIVER CANCER (PROPHETIC)

A 56 year old male patient presents with upper abdominal pain, loss of weight, loss of appetite, and jaundice. The patient is diagnosed with liver cancer and administered a therapeutically effective amount of a composition comprising an RNA aptamer having the sequence of SEQ ID NO:7. The mouse studies are used to determine a therapeutically effective administration dose, frequency, and treatment length. The patient's liver tumors are monitored during the treatment regimen. At the conclusion of the treatment regimen, the patient is evaluated and it is noted that the tumors have not grown and have decreased in size and there is no indication of tumor metastasis.

### EXAMPLE 4—METHOD OF TREATING BRAIN CANCER (PROPHETIC)

A 40 year old male patient presents with loss of balance, changes in personality and general irritability. The patient is diagnosed with brain cancer and administered a therapeutically effective amount of a composition comprising an RNA aptamer having the sequence of SEQ ID NO:7. The mouse studies are used to determine a therapeutically effective administration dose, frequency, and treatment length. The patient's brain tumor is monitored during the treatment regimen. At the conclusion of the treatment regimen, the patient is evaluated and it is noted that the tumor has not grown and decreased in size and there is no indication of tumor metastasis.

### EXAMPLE 5—METHOD OF TREATING PANCREATIC CANCER (PROPHETIC)

A 60 year old male patient presents with jaundice, back pain, and weight loss. The patient is diagnosed with pancreatic cancer and administered a therapeutically effective amount of a composition comprising an RNA aptamer having the sequence of SEQ ID NO:7. The mouse studies are used to determine a therapeutically effective administration dose, frequency, and treatment length. The patient's pancreatic tumors are monitored during the treatment regimen. At the conclusion of the treatment regimen, the patient is evaluated and it is noted that the tumors have not grown and have decreased in size and there is no indication of tumor metastasis.

### EXAMPLE 6—METHOD OF TREATING COLON CANCER (PROPHETIC)

A 65 year old female patient presents with abdominal pain, rectal bleeding, excessive gas, and constipation. The patient is diagnosed with colon cancer and administered a therapeutically effective amount of a composition comprising an RNA aptamer having the sequence of SEQ ID NO:7. The mouse studies are used to determine a therapeutically effective administration dose, frequency, and treatment length. The patient's colon tumors are monitored during the treatment regimen. At the conclusion of the treatment regimen, the patient is evaluated and it is noted that the tumors have not grown and have decreased in size and there is no indication of tumor metastasis.

### EXAMPLE 7—METHOD OF TREATING RENAL CANCER (PROPHETIC)

A 47 year old female patient presents with hematuria, fatigue, fever and weight loss. The patient is diagnosed with renal cancer and administered a therapeutically effective amount of a composition comprising an RNA aptamer having the sequence of SEQ ID NO:7. The mouse studies are used to determine a therapeutically effective administration dose, frequency, and treatment length. The patient's renal tumors are monitored during the treatment regimen. At the conclusion of the treatment regimen, the patient is evaluated and it is noted that the tumors have not grown and have decreased in size and there is no indication of tumor metastasis.

### Conclusion

An RNA aptamer targeting the extracellular sheddase domain of Adam8 was isolated and characterized both in vitro and in vivo. In co-cultures of human mesenchymal stem cells with human MDA-MB-231 breast cancer or Hep G2 cell liver cancer cells, the aptamer blocked extracellular Adam8 activities with associated reversal of the previously established cancer-derived osteopontin-induced myofibroblast cancer-associated fibroblast phenotype (myCAF). The results suggest that the signal pathways that initiate the development of the myCAF phenotype may be distinct from those required for maintenance, that extracellular Adam8 sheddase activity is required for maintenance of the myCAF phenotype, and that this aptamer may serve as a vehicle for the further investigation of this new pathway.

The sequence listing entitled “RNA Aptamer Targeting of Adam8 in Cancer Growth and Metastasis” in XML format, created on Nov. 26, 2023 and being 9,000 bytes in size, is hereby incorporated by reference into this disclosure.

### References

1. Conrad, C.; Benzel, J.; Dorzweiler, K.; Cook, L.; Schlomann, U.; Zarbock, A.; Slater, E. P.; Nimsky, C.; Bartsch, J. W. ADAM8 in invasive cancers: Links to tumor progression, metastasis, and chemoresistance. Clin. Sci. 2019, 133, 83-99.
2. Schlomann, U.; Wildeboer, D.; Webster, A.; Antropova, O.; Zeuschner, D.; Knight, C. G.; Docherty, A. J.; Lambert, M.; Skelton, L.; Jockusch, H.; et al. The Metalloprotease Disintegrin ADAM8. J. Biol. Chem. 2002, 277, 48210-48219.
3. Schlondorff, J.; Blobel, C. Metalloprotease-disintegrins: Modular proteins capable of promoting cell-cell interactions and triggering signals by protein-ectodomain shedding. J. Cell Sci. 1999, 112, 3603-3617.
4. Schlomann, U.; Koller, G.; Conrad, C.; Ferdous, T.; Golfi, P.; Garcia, A. M.; Höfling, S.; Parsons, M.; Costa, P.; Soper, R.; et al. ADAM8 as a drug target in pancreatic cancer. Nat. Commun. 2015, 6, 1-16.
5. Que-Gewirth, N. S.; A Sullenger, B. Gene therapy progress and prospects: RNA aptamers. Gene Ther. 2007, 14, 283-291.
6. Gopinath, S. C. Methods developed for SELEX. Anal. Bioanal. Chem. 2007, 387, 171-182.
7. Ireson, C. R.; Kelland, L. R. Discovery and development of anticancer aptamers. Mol. Cancer Ther. 2006, 5, 2957-2962.
8. Nimjee, S. M.; White, R. R.; Becker, R. C.; Sullenger, B. A. Aptamers as Therapeutics. Annu. Rev. Pharmacol. Toxicol. 2017, 57, 61-79.
9. Mi, Z.; Guo, H.; Russell, M. B.; Liu, Y.; A Sullenger, B.; Kuo, P. C. RNA Aptamer Blockade of Osteopontin Inhibits Growth and Metastasis of MDA-MB231 Breast Cancer Cells. Mol. Ther. 2009, 17, 153-161.
10. Cheng, L.; Huang, Z.; Zhou, W.; Wu, Q.; Donnola, S.; Liu, J. K.; Fang, X.; Sloan, A. E.; Mao, Y.; Lathia, J. D.; et al. Glioblastoma Stem Cells Generate Vascular Pericytes to Support Vessel Function and Tumor Growth. Cell 2013, 153, 139-152.
11. Murphy, M. B.; Fuller, S. T.; Richardson, P. M.; Doyle, S. A. An improved method for the in vitro evolution of aptamers and applications in protein detection and purification. Nucleic Acids Res. 2003, 31, e110.
12. Mahajan, U. M.; Li, Q.; Alnatsha, A.; Maas, J.; Orth, M.; Maier, S. H.; Peterhansl, J.; Regel, I.; Sendler, M.; Wagh, P. R.; et al. Tumor-Specific Delivery of 5-Fluorouracil-Incorporated Epidermal Growth Factor Receptor-Targeted Aptamers as an Efficient Treatment in Pancreatic Ductal Adenocarcinoma Models. Gastroenterology 2021, 161, 996-1010.e1.
13. Liao, D.-F.; Jin, Z.-G.; Baas, A. S.; Daum, G.; Gygi, S. P.; Aebersold, R.; Berk, B. C. Purification and Identification of Secreted Oxidative Stress-induced Factors from Vascular Smooth Muscle Cells. J. Biol. Chem. 2000, 275, 189-196.
14. Frances, S.; Hannah, H.; Katherine, S.; Matthew, A.; Bruno, S.; Gillian, F.; Robert, C. A detailed mammosphere assay protocol for the quantification of breast stem cell activity. J. Mammary Gland Biol. Neoplasia 2012, 17, 111-117.
15. Bhattacharya, S. D.; Mi, Z.; Kim, V. M.; Guo, H.; Talbot, L. J.; Kuo, P. C. Osteopontin Regulates Epithelial Mesenchymal Transition-Associated Growth of Hepatocellular Cancer in a Mouse Xenograft Model. Ann. Surg. 2012, 255, 319-325.
16. Rogers, M. P.; Kothari, A.; Read, M.; Kuo, P. C.; Mi, Z. Maintaining Myofibroblastic-Like Cancer-Associated Fibroblasts by Cancer Stemness Signal Transduction Feedback Loop. Cureus 2022, 14, e29354.
17. Chabata, C. V.; Frederiksen, J. W.; Sullenger, B. A.; Gunaratne, R. Emerging applications of aptamers for anticoagulation and hemostasis. Curr. Opin. Hematol. 2018, 25, 382-388.
18. Zhong, Y.; Wu, P.; He, J.; Zhong, L.; Zhao, Y. Advances of aptamer-based clinical applications for the diagnosis and therapy of cancer. Discov. Med. 2020, 29, 169-180.
19. Wu, X.; Chen, J.; Wu, M.; Zhao, J. X. Aptamers: Active Targeting Ligands for Cancer Diagnosis and Therapy. Theranostics 2015, 5, 322-344.
20. Rosenberg, J. E.; Bambury, R. M.; Van Allen, E. M.; Drabkin, H. A.; Lara, P. N.; Harzstark, A. L.; Wagle, N.; Figlin, R. A.; Smith, G. W.; Garraway, L. A.; et al. A phase II trial of AS1411 (a novel nucleolin-targeted DNA aptamer) in metastatic renal cell carcinoma. Investig. New Drugs 2014, 32, 178-187.
21. Calissano, C.; Damle, R. N.; Marsilio, S.; Yan, X.-J.; Yancopoulos, S.; Hayes, G.; Emson, C.; Murphy, E. J.; Hellerstein, M. K.; Sison, C.; et al. Intraclonal Complexity in Chronic Lymphocytic Leukemia: Fractions Enriched in Recently Born/Divided and Older/Quiescent Cells. Mol. Med. 2011, 17, 1374-1382.
22. O'connor, M. L.; Xiang, D.; Shigdar, S.; Macdonald, J.; Li, Y.; Wang, T.; Pu, C.; Wang, Z.; Qiao, L.; Duan, W. Cancer stem cells: A contentious hypothesis now moving forward. Cancer Lett. 2014, 344, 180-187.
23. Yoshida, S.; Setoguchi, M.; Higuchi, Y.; Akizuki, S.; Yamamoto, S. Molecular cloning of cDNA encoding MS2 antigen, a novel cell surface antigen strongly expressed in murine monocytic lineage. Int. Immunol. 1990, 2, 585-591.
24. Yoshiyama, K.; Higuchi, Y.; Kataoka, M.; Matsuura, K.; Yamamoto, S. CD156 (Human ADAM8): Expression, Primary Amino Acid Sequence, and Gene Location. Genomics 1997, 41, 56-62.
25. Kelly, K.; Hutchinson, G.; Nebenius-Oosthuizen, D.; Smith, A. J.; Horiuchi, K.; Rittger, A.; Manova, K.; Docherty, A. J.; Blobel, C. P. Metalloprotease-disintegrin ADAM8: Expression analysis and targeted deletion in mice. Dev. Dyn. 2005, 232, 221-231.
26. Romagnoli, M.; Mineva, N. D.; Polmear, M.; Conrad, C.; Srinivasan, S.; Loussouarn, D.; Barille-Nion, S.; Georgakoudi, I.; Dagg, Á.; McDermott, E. W.; et al. ADAM 8 expression in invasive breast cancer promotes tumor dissemination and metastasis. EMBO Mol. Med. 2014, 6, 278-294.
27. Conrad, C.; Götte, M.; Schlomann, U.; Roessler, M.; Pagenstecher, A.; Anderson, P.; Preston, J.; Pruessmeyer, J.; Ludwig, A.; Li, R.; et al. ADAM8 expression in breast cancer derived brain metastases: Functional implications on MMP-9 expression and transendothelial migration in breast cancer cells. Int. J. Cancer 2018, 142, 779-791.
28. Zhang, Y.; Tan, Y.-F.; Jiang, C.; Zhang, K.; Zha, T.-Z.; Zhang, M. High ADAM8 Expression is Associated with Poor Prognosis in Patients with Hepatocellular Carcinoma. Pathol. Oncol. Res. 2013, 19, 79-88.
29. Zhang, Y.; Zha, T. Z.; Hu, B. S.; Jiang, C.; Ge, Z. J.; Zhang, K.; Tan, Y. High expression of ADAM8 correlates with poor prognosis in hepatocellular carcinoma. Surgeon 2013, 11, 67-71.
30. Li, S. Q.; Wang, D. M.; Zhu, S.; Ma, Z.; Li, R. F.; Xu, Z. S.; Han, H. M. The important role of ADAM8 in the progression of hepatocellular carcinoma induced by diethylnitrosamine in mice. Hum. Exp. Toxicol. 2015, 34, 1053-1072.
31. Caligiuri, G.; Tuveson, D.A. Activated fibroblasts in cancer: Perspectives and challenges. Cancer Cell 2023, 41, 434-449.

The disclosures of all publications cited above are expressly incorporated herein by reference, each in its entirety, to the same extent as if each were incorporated by reference individually.

It is also to be understood that the following claims are intended to cover all of the generic and specific features of the invention herein described, and all statements of the scope of the invention which, as a matter of language, might be said to fall there between. Now that the invention has been described,

What is claimed is:

1. A composition comprising:
an RNA aptamer having at least 90% homology to SEQ ID NO:1, SEQ ID NO:2, SEQ ID NO:3, SEQ ID NO:4, SEQ ID NO:5, SEQ ID NO:6, SEQ ID NO:7, SEQ ID NO:8, or SEQ ID NO:9; and
a pharmaceutically acceptable carrier.

2. The composition of claim 1, wherein the RNA aptamer is Apt-1 having SEQ ID NO:1.

3. The composition of claim 1, wherein the RNA aptamer is Apt-1-26nt having SEQ ID NO:7.

4. A method of treating a disease characterized by upregulated Adam8 in a patient in need thereof comprising:
administering to the patient in need thereof a therapeutically effective amount of a therapeutic agent comprising an RNA aptamer having a sequence of SEQ ID NO:1, SEQ ID NO:6, SEQ ID NO:7, SEQ ID NO:8, or SEQ ID NO:9.

5. The method of claim 4, wherein the disease characterized by upregulated Adam8 is selected from the group consisting of inflammatory diseases of the lung, inflammatory diseases of the central nervous system, inflammatory diseases of the bones and joints, inflammatory diseases of the circulatory system, asthma, atherosclerosis, liver injury and cancer.

6. The method of claim 5, wherein the disease associated with upregulated Adam8 expression is a cancer.

7. The method of claim 6, wherein the cancer is selected from the group consisting of breast cancers, liver cancers, pancreatic cancers, brain cancers, colon cancers, renal cancers, bone cancers, lung cancers, and head and neck cancers.

8. The method of claim 7, wherein the cancer is breast cancer.

9. The method of claim 8, wherein the therapeutic agent administered to the patient is the RNA aptamer having the sequence of SEQ ID NO:1 or SEQ ID NO:7.

10. The method of claim 7, wherein the cancer is liver cancer.

11. The method of claim 10, wherein the therapeutic agent administered to the patient is the RNA aptamer having the sequence of SEQ ID NO:1 or SEQ ID NO:7.

12. The method of claim 4, wherein the RNA aptamer binds to a soluble extracellular metalloproteinase domain of Adam8.

13. A method of reversing a myofibroblast cancer-associated fibroblast (myCAF) phenotype in a patient in need thereof comprising:
diagnosing or having diagnosed the patient with a cancer characterized by increased expression of Adam8 as compared to a control;
determining or having determined presence of the myCAF phenotype in the patient; and
administering to the patient in need thereof a therapeutically effective amount of a therapeutic agent comprising an RNA aptamer having a sequence of SEQ ID NO:1, SEQ ID NO:6, SEQ ID NO:7, SEQ ID NO:8, or SEQ ID NO:9;
wherein the administration of the therapeutic agent reverses the myCAF phenotype in the patient.

14. The method of claim 13, wherein the cancer is selected from the group consisting of breast cancers, liver cancers, pancreatic cancers, brain cancers, colon cancers, renal cancers, bone cancers, lung cancers, and head and neck cancers.

15. The method of claim 14, wherein the cancer is breast or liver cancer.

16. The method of claim 13, wherein the myCAF phenotype is determined by an increased expression level of at least one of alpha-smooth muscle actin (α-SMA), tenascin C (TenC), vimentin A (Vim A) or a combination thereof as compared to a control.

17. The method of claim 16, wherein administration of the therapeutic agent decreases the expression level of the at least one of α-SMA, TenC, Vim A or the combination thereof to reverse the myCAF phenotype.

18. The method of claim 13, wherein the therapeutic agent administered to the patient is the RNA aptamer having the sequence of SEQ ID NO:1.

19. The method of claim 13, wherein the therapeutic agent administered to the patient is the RNA aptamer having the sequence of SEQ ID NO:7.
